(19) United States
(12) Patent Application Publication
(10) Pub. No.: US 2024/0423735 A1
(43) Pub. Date: Dec. 26, 2024

(54) METHOD AND APPARATUS FOR DRIVING A SURGICAL INSTRUMENT

(71) Applicant: LIVSMED INC., Seongnam-si (KR)

(72) Inventors: Jin Hyuk YOON, Seongnam-si (KR); Soon Yong JEONG, Seongnam-si (KR)

(21) Appl. No.: 18/750,622

(22) Filed: Jun. 21, 2024

(30) Foreign Application Priority Data
Jun. 23, 2023 (KR) 10-2023-0080837

(51) Int. Cl. A61B 34/37; G16H 20/40; G16H 40/67; G16H 70/20

(52) U.S. Cl. CPC A61B 34/37; G16H 20/40; G16H 40/67; G16H 70/20

(57) ABSTRACT

The present disclosure relates to a method and apparatus for driving surgical instruments. The method according to an embodiment of the present disclosure may generate manipulation information related to a user's motion for driving a surgical instrument, calculate first driving information based on the manipulation information, determine the presence of a risk associated with a motion of the surgical instrument based on the first driving information, and drive the surgical instrument based on the result of determining the presence of the risk.

### CROSS-REFERENCE TO RELATED APPLICATION

This application is based on and claims priority under 35 USC § 119 to Korean Patent Application No. 10-2023-0080837, filed on Jun. 23, 2023, in the Korean Intellectual Property Office, the disclosure of which is incorporated by reference herein in its entirety.

### BACKGROUND

### 1. Field

The present disclosure relates to a method and apparatus for driving surgical instruments.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

Here, a surgical robot refers to a robot that has a function of replacing a surgical action performed by a surgeon. Compared to humans, the surgical robot has the advantage of being able to operate with greater accuracy and precision, as well as being able to operate remotely.

Meanwhile, a surgical robot is generally composed of a master robot and a slave robot. When a surgical operator manipulates a control lever (e.g., a handle) equipped on the master robot, a surgical tool coupled to or held by a robot arm equipped on the slave robot may be manipulated to perform surgery.

However, several problems may arise due to the fact that the surgical operator performs the surgery by remotely manipulating the surgical tool through the surgical robot rather than physically manipulating the surgical tool directly. For example, even when the surgical operator manipulates the control lever equipped on the master robot, the slave robot may not perform motions desired by the surgical operator due to mechanical constraints. In addition, even when the slave robot performs the motions desired by the surgical operator, as the surgical operator manipulates the control lever equipped on the master robot, the motions of the slave robot may cause damage to the surgical robot or the human body of the patient of the surgery.

The aforementioned background technology is technical information possessed by the inventor for derivation of the present disclosure or acquired by the inventor during the derivation of the present disclosure, and is not necessarily prior art disclosed to the public before the application of the present disclosure.

### SUMMARY

The present disclosure is directed to providing a method and apparatus for driving surgical instruments. The present disclosure is also directed to providing a computer-readable recording medium having recorded thereon a program for executing the method on a computer.

The problem to be solved by the present disclosure is not limited to the problems mentioned above, and other problems and advantages of the present disclosure, which are not mentioned, will be understood by the following description, and will be more clearly understood by the embodiments of the present disclosure. In addition, it will be appreciated that the problems and advantages to be solved by the present disclosure may be realized by means and combinations thereof indicated in the claims.

Additional aspects will be set forth in part in the description which follows and, in part, will be apparent from the description, or may be learned by practice of the presented embodiments of the disclosure.

As a technical means for achieving the above-described technical problem, a first aspect of the present disclosure may provide a method of driving a surgical instrument, the method including generating manipulation information related to a motion of a user for driving the surgical instrument, calculating first driving information based on the manipulation information, determining a presence of a risk associated with a motion of the surgical instrument based on the first driving information, and driving the surgical instrument based on a result of the determining the presence of the risk.

In the first aspect, the generating of the manipulation information may include generating the manipulation information based on position and orientation information of a member that allows the user to manipulate a position and a function of the surgical instrument.

In the first aspect, the calculating of the first driving information may include calculating intermediate driving information based on the manipulation information and a transformation relationship between the motion of the user and the motion of the surgical instrument, and calculating the first driving information based on the intermediate driving information and a preset transformation ratio of the motion of the user to the motion of the surgical instrument.

In the first aspect, the calculating of the intermediate driving information may include calculating intermediate position information of the surgical instrument based on a transformation relationship between a member, which allows the user to manipulate a position and a function of the surgical instrument, and a camera attached to the surgical instrument, calculating intermediate orientation information of the surgical instrument based on a transformation relationship between the member and the surgical instrument, and generating the intermediate driving information using the intermediate position information of the surgical instrument and the intermediate orientation information of the surgical instrument.

In the first aspect, the calculating of the first driving information may include calculating position information of the surgical instrument based on intermediate position information of the surgical instrument included in the intermediate driving information and a preset position transformation ratio, calculating orientation information of the surgical instrument based on intermediate orientation information of the surgical instrument included in the intermediate driving information and a preset orientation transformation ratio, and generating the first driving information using the position information of the surgical instrument and the orientation information of the surgical instrument.

In the first aspect, the determining of the presence of a risk includes determining the presence of the risk based on at least one of whether the range of motion of the surgical instrument has exceeded a preset threshold value and whether the surgical instrument corresponds to a preset singularity region.

In the first aspect, when the presence of the risk is determined based on the range of motion of the surgical instrument, the determining of the presence of the risk may include calculating expected driving information based on initial driving information of the surgical instrument and the motion range information of the surgical instrument, calculating reference driving information based on the motion range information, and determining the presence of the risk associated with the range of motion of the surgical instrument based on the expected driving information, the motion range information, and the first driving information.

In a first aspect, when the presence of a risk is determined based on the preset singularity region, the determining of the presence of the risk may include calculating manipulability information related to the first driving information and manipulability information related to the preset singularity region based on the first driving information, and determining the presence of the risk associated with the preset singularity region of the surgical instrument based on the manipulability information related to the first driving information and the manipulability information related to the preset singularity region.

In the first aspect, the method may further include updating the first driving information based on the result of determining the presence of the risk.

In the first aspect, the updating of the first driving information may include updating the first driving information based on motion range information of the surgical instrument.

In the first aspect, the updating of the first driving information may include generating compensation driving information based on initial driving information of the surgical instrument, the first driving information, and vector information related to a singularity region, and updating the first driving information based on the initial driving information of the surgical instrument, the first driving information, manipulability information related to the first driving information, manipulability information related to the singularity region, and the compensation driving information.

In the first aspect, the updating of the first driving information may include updating the first driving information by changing only orientation information of the surgical instrument included in the first driving information.

In the first aspect, the driving of the surgical instrument may include calculating second driving information based on the first driving information, and driving the surgical instrument based on the second driving information.

A second aspect of the present disclosure may provide a method of driving a surgical instrument, the method including generating manipulation information corresponding to manipulation of a user input part for driving a multi-joint type surgical instrument, calculating first driving information based on the manipulation information, determining a presence of a risk associated with a motion of the multi-joint type surgical instrument based on the first driving information, and driving the multi-joint type surgical instrument based on a result of determining the presence of the risk.

In the second aspect, the generating of the manipulation information may include generating the manipulation information based on position and orientation information of the user input part of the multi-joint type surgical instrument.

In the second aspect, the calculating of the first driving information may include calculating intermediate driving information based on the manipulation information, a transformation relationship between the user input part and the multi-joint type surgical instrument, a transformation relationship between the user input part and an end tool, and a transformation relationship between the user input part and a camera attached to the multi-joint type surgical instrument, and calculating the first driving information based on the intermediate driving information and a preset transformation ratio of a motion of the user input part to a motion of the multi-joint type surgical instrument.

In the second aspect, the calculating of the intermediate driving information may include calculating intermediate position information of the multi-joint type surgical instrument based on the transformation relationship between the user input part and the camera attached to the multi-joint type surgical instrument, calculating intermediate orientation information of the surgical instrument based on the transformation relationship between the user input part and the multi-joint type surgical instrument, and generating the intermediate driving information using the intermediate position information of the multi-joint type surgical instrument and the intermediate orientation information of the multi-joint type surgical instrument.

In the second aspect, the calculating of the first driving information may include calculating position information of the multi-joint type surgical instrument based on intermediate position information of the multi-joint type surgical instrument included in the intermediate driving information and a preset position transformation ratio, calculating orientation information of the multi-joint type surgical instrument based on intermediate orientation information of the multi-joint type surgical instrument included in the intermediate driving information and a preset orientation transformation ratio, and generating the first driving information using the position information of the multi-joint type surgical instrument and the orientation information of the multi-joint type surgical instrument.

In the second aspect, the determining of the presence of the risk may include determining the presence of the risk based on at least one of whether a range of motion formed by at least one joint of the multi-joint type surgical instrument has exceeded a preset threshold value and whether the multi-joint type surgical instrument corresponds to a preset singularity region.

In the second aspect, when the presence of the risk is determined based on the range of motion of the multi-joint type surgical instrument, the determining of the presence of the risk may include calculating expected driving information based on initial driving information of the multi-joint type surgical instrument and motion range information of the multi-joint type surgical instrument, calculating reference driving information based on the motion range information, and determining the presence of the risk associated with the range of motion formed by at least one joint of the multi-joint type surgical instrument based on the expected driving information, the motion range information, and the first driving information.

In the second aspect, when the presence of the risk is determined based on the preset singularity region, the determining of the presence of the risk may include calculating manipulability information related to the first driving information and manipulability information related to the preset singularity region based on the first driving information, and determining the presence of the risk associated with the preset singularity region of the multi-joint type surgical instrument based on the manipulability information related to the first driving information and the manipulability information related to the preset singularity region.

In the second aspect, the method may further include updating the first driving information based on the result of determining the presence of the risk.

In the second aspect, the updating of the first driving information may include updating the first driving information based on motion range information of the multi-joint type surgical instrument.

In the second aspect, the updating of the first driving information may include generating compensation driving information based on initial driving information of the multi-joint type surgical instrument, the first driving information, and vector information related to a singularity region, and updating the first driving information based on the initial driving information of the multi-joint type surgical instrument, the first driving information, manipulability information related to the first driving information, manipulability information related to the singularity region, and the compensation driving information.

In the second aspect, the updating of the first driving information may include updating the first driving information by changing only orientation information of the multi-joint type surgical instrument included in the first driving information.

In the second aspect, the driving of the surgical instrument may include calculating second driving information based on the first driving information, and driving the multi-joint type surgical instrument based on the second driving information.

A third aspect of the present disclosure may provide a computing device including at least one memory, and at least one processor, wherein the at least one processor is configured to generate manipulation information related to a motion of a user for driving a surgical instrument, calculate first driving information based on the manipulation information, determine a presence of a risk associated with a motion of the surgical instrument based on the first driving information, and drive the surgical instrument based on a result of determining the presence of the risk.

A fourth aspect of the present disclosure may provide a non-transitory computer-readable recording medium having recorded thereon a program for executing the method according to the first aspect on a computer.

In addition, other methods and systems for implementing the present disclosure, and a non-transitory computer-readable recording medium having recorded thereon a program for executing the method may be further provided.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for describing an example of a system for driving a surgical instrument according to an embodiment.

FIG. 2A is a configuration diagram illustrating an example of a user terminal according to an embodiment.

FIG. 2B is a configuration diagram illustrating an example of a server according to an embodiment.

FIG. 3 is a diagram for describing another example of the system for driving a surgical instrument according to an embodiment.

FIG. 4 is a block diagram illustrating an internal configuration of the surgical robot system of FIG. 3.

FIG. 5 is a perspective view illustrating a slave robot of the surgical robot system of FIG. 3 and a multi-joint type surgical instrument mounted on the slave robot.

FIG. 6 is a perspective view illustrating a multi-joint type surgical instrument according to an embodiment of the present disclosure.

FIGS. 7 and 8 are perspective views of an end tool of the multi-joint type surgical instrument of FIG. 6.

FIG. 9 is a plan view of the end tool of the multi-joint type surgical instrument of FIG. 6.

FIGS. 10 and 11 are perspective views of a driving part of the multi-joint type surgical instrument of FIG. 6

FIG. 12 is a plan view of the driving part of the multi-joint type surgical instrument of FIG. 6.

FIG. 13 is a rear view of the driving part of the multi-joint type surgical instrument of FIG. 6.

FIG. 14 is a side view of the driving part of the multi-joint type surgical instrument of FIG. 6.

FIG. 15 is a view illustrating the configuration of pulleys and wires of the multi-joint type surgical instrument illustrated in FIG. 6, in detail for the configuration related to a first jaw.

FIG. 16 is a view illustrating the configuration of pulleys and wires of the multi-joint type surgical instrument illustrated in FIG. 6, in detail for the configuration related to a second jaw.

FIGS. 17 and 18 are views illustrating a pitch motion of the multi-joint type surgical instrument illustrated in FIG. 6.

FIGS. 19 and 20 are views illustrating a yaw motion of the multi-joint type surgical instrument illustrated in FIG. 6.

FIG. 21 is a flowchart for describing an example of a method of driving a surgical instrument according to an embodiment.

FIG. 22 is a flowchart for describing an example of a method by which a processor according to an embodiment calculates first driving information.

FIG. 23 is a view for describing an example of a transformation relationship between a motion of a user and a motion of a surgical instrument according to an embodiment.

FIG. 24 is a view for describing an example of the surgical instrument operating based on the first driving information according to an embodiment.

FIG. 25 is a view for describing an example of a range of motion of the surgical instrument according to an embodiment.

FIG. 26 is a flowchart for describing another example of the method of driving the surgical instrument according to an embodiment.

FIG. 27 is a flowchart for describing another example of the method by which the processor calculates the first driving information according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, various embodiments of the present disclosure are described with reference to the accompanying drawings While the present disclosure is susceptible to various modifications and may have several embodiments, specific embodiments thereof are shown by way of example in the drawings and will herein be described in detail. However, it should be understood that there is no intent to limit the present disclosure to the specific embodiments, but on the contrary, the present disclosure is to cover all modifications, equivalents, and alternatives falling within the spirit and scope of the present disclosure. With regard to description of the drawings, like reference numerals have been used for like components.

Expressions such as “includes” or “may include” that may be used in various embodiments of the present disclosure indicate the existence of a corresponding function, operation, or component that is disclosed, and are not intended to limit one or more additional functions, operations, or components. In addition, in the various embodiments of the present disclosure, it is to be understood that the terms such as “including,” “having,” and the like are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

In various embodiments of the present disclosure, the expression “or” includes any and all combinations of one or more of the associated listed items. For example, “A or B” may include “A,” “B,” or “both A and B.”

While expressions such as “first” and “second” used in the various embodiments of the present disclosure may describe various components of the various embodiments, the corresponding components are not limited by the expressions such as “first” and “second.” For example, these expressions do not limit the order and/or importance of corresponding components. These expressions may be used to distinguish one component from another. For example, both a first user device and a second user device are user devices and indicate different user devices. For example, a first component may be named a second component or a second component may be named a first component without departing from the scope of the various embodiments of present disclosure.

In an embodiment of the present disclosure, the terms “module,” “unit,” “part,” or the like are terms which designate a component that performs at least one function or operation, and the component may be implemented with a hardware or software, or a combination of hardware and software. In addition, a plurality of “modules,” a plurality of “units,” or a plurality of “parts”, except for “a module,” “a unit,” or a “part” which needs to be implemented to a specific hardware, may be integrated to at least one module or a chip and implemented in at least one processor.

The terms used in various embodiments of the present disclosure are used to describe a particular embodiment only and are not intended to limit the various embodiments of the present disclosure. Singular forms are intended to include plural forms as well, unless the context clearly indicates otherwise.

Unless otherwise defined, all terms used herein, including technical or scientific terms, have the same meanings as those generally understood by those with ordinary knowledge in the field of art to which the various embodiments of the present disclosure belongs.

Generally used terms defined in a dictionary should be interpreted to have meanings the same as meanings in the context of the related art and are not interpreted as ideal or excessively formal meanings unless the various embodiments of the present disclosure clearly define otherwise.

Hereinafter, various embodiments of the present disclosure will be described in detail with reference to the accompanying drawings.

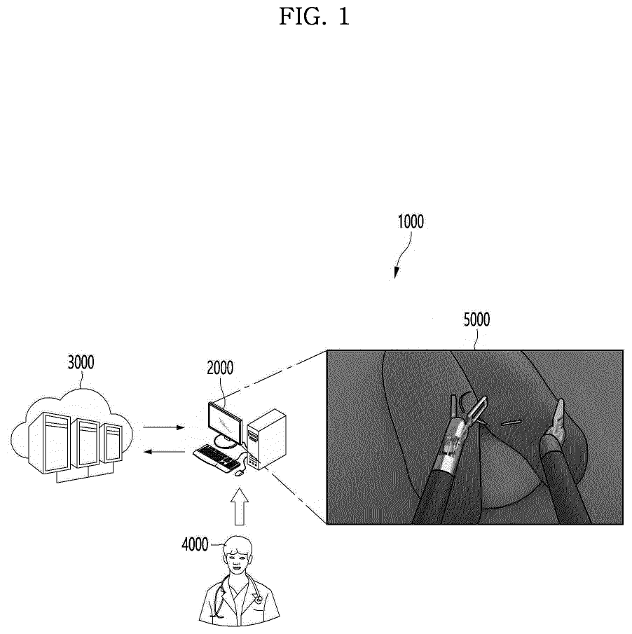

FIG. 1 is a diagram for describing an example of a system for driving a surgical instrument according to an embodiment.

Referring to FIG. 1, a system 1000 includes a user terminal 2000 and a server 3000. For example, the user terminal 2000 and the server 3000 may be connected to each other by a wired or wireless communication method to transmit and receive data (e.g., manipulation information, first driving information, intermediate driving information, second driving information, the presence of a risk associated with a motion of a surgical instrument, or the like) to and from each other.

For convenience of description, in FIG. 1, the system 1000 is illustrated as including the user terminal 2000 and the server 3000, but the present disclosure is not limited thereto. For example, the system 1000 may include another external device (not shown), and operations of the user terminal 2000 and the server 3000 to be described below may be implemented by a single device (e.g., the user terminal 2000 or the server 3000) or a plurality of devices.

The user terminal 2000 may include a display device and a device for receiving user input (e.g., a keyboard, a mouse, or the like), and may be a computing device including a memory and a processor. For example, the display device may be implemented as a touch screen and may receive user input. For example, the user terminal 2000 may correspond to a notebook personal computer (PC), a desktop PC, a laptop computer, a tablet computer, a smartphone, and the like, but the present disclosure is not limited thereto.

The server 3000 may be a device that communicates with an external device (not shown) including the user terminal 2000. In an example, the server 3000 may be a device for storing various pieces of data including manipulation information related to a motion of a user, first driving information related to a motion of a surgical instrument, the presence of a risk associated with the motion of the surgical instrument, and the like.

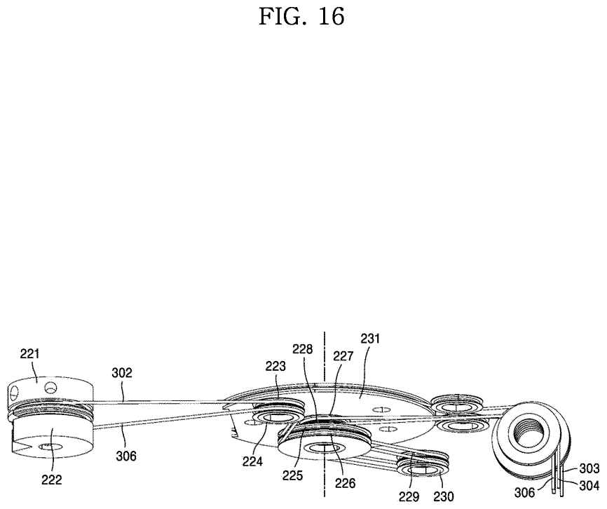

Alternatively, the server 3000 may be a computing device that includes memory and a processor and has its own computing capabilities. For example, the server 3000 may perform at least some of operations of the user terminal 2000 to be described later with reference to FIGS. 1 to 27. For example, the server 3000 may be a cloud server, but the present disclosure is not limited thereto.

The user terminal 2000 may calculate first driving information related to a motion of the surgical instrument by using manipulation information related to a user's motion for driving the surgical instrument, and determine the presence of a risk associated with the motion of the surgical instrument based on the first driving information, thereby driving the surgical instrument. For example, the user terminal 2000 may generate manipulation information related to a user's motion for driving the surgical instrument. In addition, the user terminal 2000 may calculate first driving information based on the manipulation information. In addition, the user terminal 2000 may determine the presence of a risk associated with a motion of the surgical instrument based on the first driving information. In addition, the user terminal 2000 may drive the surgical instrument based on the result of determining the presence of the risk.

The manipulation information refers to information indicating a user's motion for driving the surgical instrument. For example, the manipulation information may include position information and orientation information, in a three-dimensional coordinate system, of a member that allows the position and function of the surgical instrument to be manipulated by the user's motion.

The first driving information refers to information indicating the motion of the surgical instrument to move its position and orientation as intended by the user, in response to the manipulation information, For example, the first driving information may include one or more of position information and orientation information of the surgical instrument, which is calculated based on the position or orientation information included in the manipulation information and a transformation relationship between the user's motion and the motion of the surgical instrument.

A presence of a risk exists when the surgical instrument may not operate in response to a user's motion due to mechanical constraints while being operated in response to the user's motion, or there is a risk of causing damage to the surgical instrument or to the human body, which is a patient of the surgery, even when the surgical instrument operates in response to the user's motion.

Meanwhile, the user terminal 2000 may update the first driving information based on the result of determining the presence of the risk. In an example, when driving the surgical instrument based on the first driving information is determined to be dangerous as the result of determining the presence of the risk, the user terminal 2000 may update the first driving information. In another example, when driving the surgical instrument based on the first driving information is determined not to be dangerous as the result of determining the presence of the risk, the user terminal 2000 may drive the surgical instrument based on the first driving information without updating the first driving information.

Meanwhile, the user terminal 2000 may calculate second driving information based on the first driving information, and may drive the surgical instrument based on the second driving information. For example, the user terminal 2000 may calculate the second driving information by performing an inverse kinematic transformation based on the first driving information, and may drive the surgical instrument based on the calculated second driving information.

The second driving information refers to information related to an angle of a joint of the surgical instrument with respect to the motion of the surgical instrument. For example, the second driving information may include information related to an angle of the joint of the surgical instrument required for the surgical instrument to operate in response to the user's motion by using the position or orientation information of the surgical instrument included in the first driving information. As will be described below, the second driving information may be obtained based on the first driving information.

For example, the user terminal 2000 may determine the presence of a risk associated with a motion of the surgical instrument, or drive the surgical instrument through an application installed in the user terminal 2000. Here, the application may be a software program installed for the activities of a user 4000 to drive the surgical instrument. For example, through the application, the user 4000 may perform various activities such as generating manipulation information related to a user's motion for driving the surgical instrument, calculating first driving information based on the manipulation information, determining the presence of a risk associated with a motion of the surgical instrument based on the first driving information, driving the surgical instrument based on the result of determining the presence of the risk, and updating the first driving information based on the result of determining the presence of the risk.

Meanwhile, the user terminal 2000 may output an image 5000 indicating the motion of the surgical instrument driven based on the motion of the user 4000. For example, the user terminal 2000 may generate manipulation information related to a motion of the user 4000 necessary for the user 4000 to drive the surgical instrument. In addition, the user terminal 2000 may generate first driving information based on the manipulation information. In addition, the user terminal 2000 may determine the presence of a risk associated with a motion of the surgical instrument based on the first driving information. In addition, the user terminal 2000 may drive the surgical instrument based on the result of determining the presence of the risk. In addition, the user terminal 2000 may output the image 5000 representing the motion of the surgical instrument driven using the first driving information. The image 5000, representing a motion of the surgical instrument, allows the user 4000 to intuitively understand the motion of the surgical instrument in relation to the motion of the user 4000, and to more accurately manipulate the surgical instrument.

Meanwhile, for convenience of description, it has been described throughout the specification that the user terminal 2000 generates manipulation information related to a user's motion for driving the surgical instrument, generates first driving information based on the manipulation information, determines the presence of a risk associated with a motion of the surgical instrument based on the first driving information, and drives the surgical instrument based on the result of determining the presence of the risk, but the present disclosure is not limited thereto. For example, at least some of operations performed by the user terminal 2000 may be performed by the server 3000.

In other words, at least some of operations of the user terminal 2000 to be described with reference to FIGS. 1 to 27 may be performed by the server 3000. For example, the server 3000 may generate manipulation information related to a user's motion for driving the surgical instrument, generate first driving information based on the manipulation information, determine the presence of a risk associated with a motion of the surgical instrument based on the first driving information, and drive the surgical instrument based on the result of determining the presence of the risk. In addition, the server 3000 may update the first driving information based on the result of determining the presence of the risk.

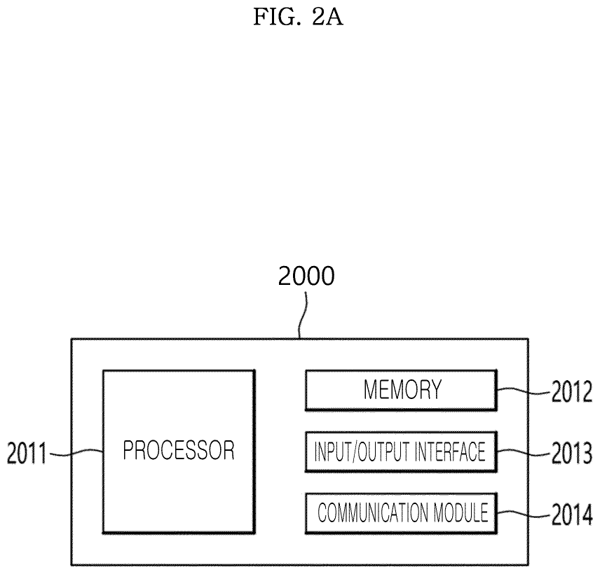

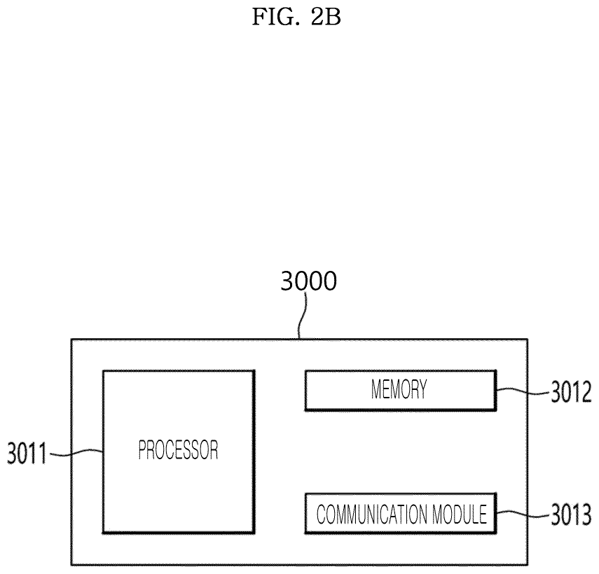

FIG. 2A is a configuration diagram illustrating an example of the user terminal according to an embodiment.

Referring to FIG. 2A, a user terminal 2000 includes a processor 2011, a memory 2012, an input/output interface 2013, and a communication module 2014. For convenience of description, only components related to the present disclosure are illustrated in FIG. 2A. Accordingly, other general-purpose components in addition to the components illustrated in FIG. 2A may be further included in the user terminal 2000. In addition, it will be apparent to those skilled in the art related to the present disclosure that the processor 2011, the memory 2012, the input/output interface 2013, and the communication module 2014 illustrated in FIG. 2A may be implemented as independent devices.

The processor 2011 may process instructions of a computer program by performing a basic arithmetic operation, a logic operation, and an input/output operation. Here, the instructions may be provided from the memory 2012 or an external device (e.g., the server 3000 or the like). In addition, the processor 2011 may control overall operations of the other components included in the user terminal 2000.

The processor 2011 generates manipulation information related to a user's motion for driving the surgical instrument. For example, the processor 2011 may generate the manipulation information related to the user's motion based on a member that allows the position and function of the surgical instrument to be manipulated by the user's motion.

The member that allows the position and function of the surgical instrument to be manipulated by the user's motion may be a member provided in the form of a handle-shaped manipulation member, but is not limited thereto, and may be implemented in many different forms to achieve the same purpose. For example, a portion of the member may be provided in the form of a handle, and the other portions thereof may be provided in different forms, such as a clutch button. In addition, a finger insertion tube may be further formed so as to allow the surgical operator's finger to be inserted therethrough and fixed to facilitate manipulation of the surgical tool.

The manipulation information refers to information indicating a user's intuitive motion to manipulate the position and function of the surgical instrument. Specifically, the manipulation information may include position information and orientation information, in a physical coordinate system, of the member that allows a user to manipulate the position and function of the surgical instrument. In an example, the manipulation information may include a transform matrix representing linear and rotational movements in a homogeneous coordinate system. The transform matrix may be a homogeneous transform matrix, or may include information related to a rotation matrix and information related to a translation vector. In another example, the manipulation information may include position information and orientation information in a physical coordinate system represented by a representation method such as a screw. However, examples of the manipulation information are not limited to those described above.

Meanwhile, the processor 2011 may generate manipulation information based on position information and orientation information of a member that allows a user to manipulate the position and function of the surgical instrument. For example, the processor 2011 may generate the manipulation information using a difference between initial position and orientation information of the member that allows a user to manipulate the position and function of the surgical instrument, and position and orientation information of the member after the user's motion.

In addition, the processor 2011 calculates first driving information based on the manipulation information. For example, the processor 2011 may calculate first driving information related to a motion of a multi-joint type surgical instrument based on the manipulation information.

Meanwhile, in calculating the first driving information, the processor 2011 may calculate intermediate driving information based on the manipulation information and a transformation relationship between the motion of the user and the motion of the surgical instrument, and calculate the first driving information based on the intermediate driving information and a preset transformation ratio of the user's motion to the motion of the surgical instrument.

Here, the intermediate driving information refers to information calculated by transforming the motion of the user into the motion of the surgical instrument based on the manipulation information. For example, the intermediate driving information may include information calculated without considering the transformation ratio of the user's motion to the motion of the surgical instrument.

According to an embodiment of the present disclosure, by calculating the first driving information based on the intermediate driving information and the preset transformation ratio of the user's motion to the motion of the surgical instrument, it is possible to operate the surgical instrument by reflecting user's detailed manipulation activities, and help with precision surgery.

Meanwhile, in calculating the intermediate driving information, the processor 2011 may calculate intermediate position information of the surgical instrument based on a transformation relationship between the member, which allows a user to manipulate the position and function of the surgical instrument, and a camera attached to the surgical instrument, calculate intermediate orientation information of the surgical instrument based on a transformation relationship between the member and the surgical instrument, and generate intermediate driving information using the intermediate position information and the intermediate orientation information of the surgical instrument.

Meanwhile, in calculating the first driving information, the processor 2011 may calculate position information of the surgical instrument based on the intermediate position information of the surgical instrument included in the intermediate driving information and a preset position transformation ratio, calculate orientation information of the surgical instrument based on the intermediate orientation information of the surgical instrument included in the intermediate driving information and a preset orientation transformation ratio, and generate the first driving information using the position information and the orientation information of the surgical instrument.

In addition, the processor 2011 may determine the presence of a risk associated with a motion of the surgical instrument based on the first driving information. In an example, when driving the surgical instrument based on the first driving information, the processor 2011 may determine the presence of a risk such as exceeding an operable range of the surgical instrument. In another example, when driving the surgical instrument based on the first driving information, the processor 2011 may determine the presence of a risk such as an expected motion of the surgical instrument falling with a preset singularity region.

Meanwhile, in determining the presence of a risk, the processor 2011 may determine the presence of a risk based on at least one of whether a range of motion of the surgical instrument exceeds a preset threshold value and whether the surgical instrument corresponds to a preset singularity region.

Meanwhile, in determining the presence of a risk, when the processor 2011 determines the presence of a risk based on the range of motion, the processor 2011 may calculate expected driving information based on initial driving information of the surgical instrument and motion range information of the surgical instrument, calculate reference driving information based on the motion range information, and determine the presence of a risk associated with the range of motion of the surgical instrument based on the expected driving information, the motion range information, and the first driving information.

Meanwhile, in determining the presence of a risk, when the processor 2011 determines the presence of a risk based on the singularity region, the processor 2011 may calculate manipulability information related to the first driving information and manipulability information related to the singularity region based on the first driving information, determine the presence of a risk associated with the singularity region of the surgical instrument based on the manipulability information related to the first driving information and the manipulability information related to the singularity region.

Meanwhile, the processor 2011 may update the first driving information based on the result of determining the presence of the risk. In an example, when the processor 2011 drives the surgical instrument based on the first driving information, the processor 2011 may update the first driving information when it is determined that the range of motion of the surgical instrument exceeds the preset threshold value and thus it is dangerous. In another example, when the processor 2011 drives the surgical instrument based on the first driving information, the processor 2011 may update the first driving information when it is determined that the range of motion of the surgical instrument corresponds to the preset singularity region and thus it is dangerous.

Meanwhile, in updating the first driving information, the processor 2011 may update the first driving information based on the motion range information of the surgical instrument.

Meanwhile, in updating the first driving information, the processor 2011 may generate compensation driving information based on the initial driving information, the first driving information, and vector information related to the singularity region of the surgical instrument, and update the first driving information based on the initial driving information of the surgical instrument, the first driving information, the manipulability information related to the first driving information, the manipulability information related to the singularity region, and the compensation driving information.

Meanwhile, in updating the first driving information, the processor 2011 may update the first driving information by changing only the orientation information of the surgical instrument included in the first driving information. For example, the processor 2011 may update the first driving information by fixing the position information of the surgical instrument included in the first driving information and changing only the orientation information.

According to an embodiment of the present disclosure, by changing only the orientation information of the surgical instrument included in the first driving information, a situation that causes a kinematic risk and a situation that causes damage to the human body due to the motion of the surgical instrument can be prevented in advance.

In addition, the processor 2011 may drive the surgical instrument based on the result of determining the presence of the risk. In an example, when driving the surgical instrument based on the first driving information is determined to be dangerous, the processor 2011 may not drive the surgical instrument based on the first driving information. In another example, when driving the surgical instrument based on the first driving information is determined not to be dangerous, the processor 2011 may drive the surgical instrument based on the first driving information. In another example, when driving the surgical instrument based on the first driving information is determined to be dangerous, the processor 2011 may not drive the surgical instrument based on the first driving information and may update the first driving information as will be described below.

Meanwhile, in driving the surgical instrument, the processor 2011 may calculate second driving information based on the first driving information, and may drive the surgical instrument based on the second driving information. For example, the processor 2011 may calculate the second driving information by performing an inverse kinematic transformation based on the first driving information, and may drive the surgical instrument based on the calculated second driving information.

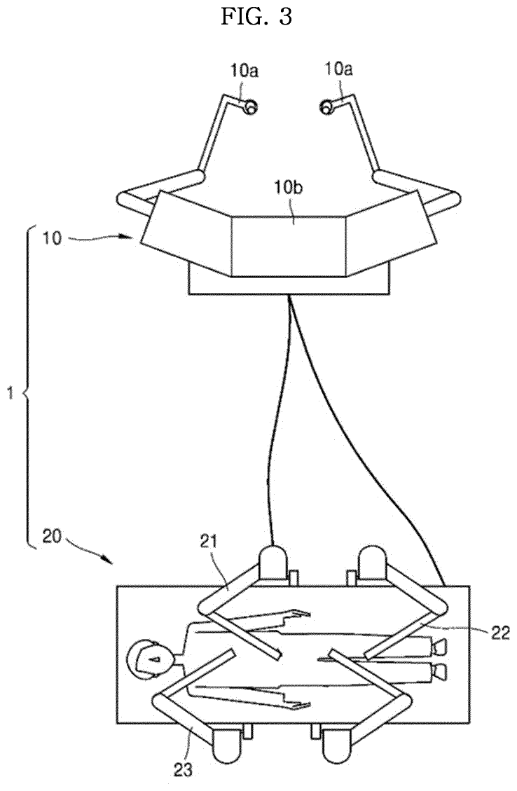

Specific examples in which the processor 2011 according to an embodiment operates will be described with reference to FIGS. 3 to 27.

The processor 2011 may be implemented in an array of multiple logic gates, or in a combination of a universal microprocessor and a memory that stores a program executable in the microprocessor. For example, the processor 2011 may include a general-purpose processor, a central processing unit (CPU), a microprocessor, a digital signal processor (DSP), a controller, a microcontroller, a state machine, or the like. In some environments, the processor 2011 may include an application-specific semiconductor (ASIC), a programmable logic device (PLD), a field programmable gate array (FPGA), or the like. For example, the processor 2011 may refer to a combination of processing devices such as, for example, a combination of a DSP and a microprocessor, a combination of a plurality of microprocessors, a combination of one or more microprocessors in conjunction with a DSP core, or a combination of any other such configuration.

The memory 2012 may include any non-transitory computer-readable recording medium. In an example, the memory 2012 may include a permanent mass storage device such as a random access memory (RAM), a read-only memory (ROM), a disk drive, a solid state drive (SSD), a flash memory, or the like. In another example, the permanent mass storage device such as a ROM, SSD, a flash memory, a disk drive, or the like may be a separate permanent storage device which is distinguishable from the memory. In addition, an operating system (OS) and at least one program code (e.g., a code for the processor 2011 to perform operations to be described later with reference to FIGS. 3 to 27) may be stored in the memory 2012.

These software components may be loaded from a computer-readable recording medium separate from the memory 2012. The separate computer-readable recording medium may be a recording medium that may be directly connected to the user terminal 2000, and may include, for example, a computer-readable recording medium, such as a floppy drive, a disk, a tape, a DVD/CD-ROM drive, a memory card, or the like. Alternatively, the software components may be loaded into the memory 2012 through the communication module 2014 instead of the computer-readable recording medium. For example, at least one program may be loaded into the memory 2012 based on a computer program (for example, a computer program for performing, by the processor 2011, operations to be described later with reference to FIGS. 3 to 27) installed by the files provided through the communication module 2014 by developers or a computer file distribution system that distributes the installation files of applications.

The input/output interface 2013 may be a member for an interface with a device (e.g., a keyboard, a mouse, or the like) for input or output, the member being connected to the user terminal 2000 or being included in the user terminal 2000. The input/output interface 2013 may be configured separately from the processor 2011, but the present disclosure is not limited thereto, and the input/output interface 2013 may be configured to be included in the processor 2011.

The communication module 2014 may provide a configuration or a function for the server 3000 and the user terminal 2000 to communicate with each other through a network. In addition, the communication module 2014 may provide a configuration or function for the user terminal 2000 to communicate with another external device. For example, a control signal, a command, data, or the like, which is provided according to the control of the processor 2011, may be transmitted to the server 3000 and/or an external device through the communication module 2014 and the network.

Meanwhile, although not shown in FIG. 2A, the user terminal 2000 may further include a display device. For example, the display device may be implemented as a touch screen. Alternatively, the user terminal 2000 may be connected to an independent display device through a wired or wireless communication method to transmit/receive data to or from each other. For example, a video, an image, or the like of driving the surgical instrument may be provided through the display device by using driving information.

FIG. 2B is a configuration diagram illustrating an example of the server according to an embodiment.

Referring to FIG. 2B, a server 3000 includes a processor 3011, a memory 3012, and a communication module 3013. For convenience of description, only components related to the present disclosure are illustrated in FIG. 2B. Accordingly, other general-purpose components other than the components illustrated in FIG. 2B may be further included in the server 3000. In addition, it will be apparent to those skilled in the art related to the present disclosure that the processor 3011, the memory 3012, and the communication module 3013 illustrated in FIG. 2B may be implemented as independent devices.

The processor 3011 may generate manipulation information related to a user's motion for driving the surgical instrument. In addition, the processor 3011 may calculate first driving information based on the manipulation information. In addition, the processor 3011 may determine the presence of a risk associated with a motion of the surgical instrument based on the first driving information. In addition, the processor 3011 may drive the surgical instrument based on the result of determining the presence of the risk. In addition, the processor 2011 may update the first driving information based on the result of determining the presence of the risk.

In other words, at least one of the operations of the processor 2011 described above with reference to FIG. 2A may be performed by the processor 3011. In this case, the user terminal 2000 may output information transmitted from the server 3000 through the display device.

Meanwhile, an implementation example of the processor 3011 is the same as the implementation example of the processor 2011 described above with reference to FIG. 2A, and thus a detailed description thereof will be omitted.

Various data such as data required for an operation of the processor 3011 and data generated according to the operation of the processor 3011 may be stored in the memory 3012. In addition, an operating system (OS) and at least one program (e.g., a program necessary for the operation of the processor 3011, or the like) may be stored in the memory 3012.

Meanwhile, an implementation example of the memory 3012 is the same as the implementation example of the memory 2012 described above with reference to FIG. 2A, and thus a detailed description thereof will be omitted.

The communication module 3013 may provide a configuration or function for the server 3000 and the user terminal 2000 to communicate with each other through a network. In addition, the communication module 2014 may provide a configuration or function for the server 3000 to communicate with another external device. For example, a control signal, a command, data, or the like, which is provided according to the control of the processor 3011, may be transmitted to the user terminal 2000 and/or an external device through the communication module 3013 and the network.

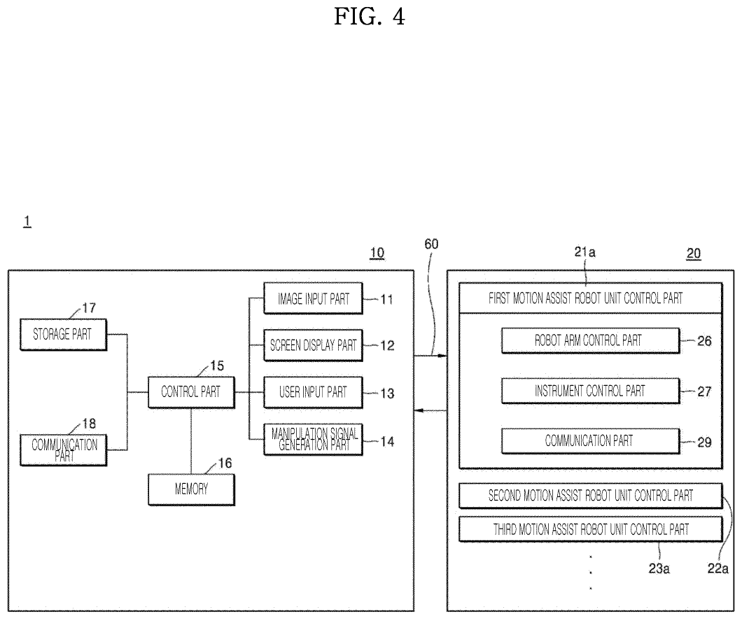

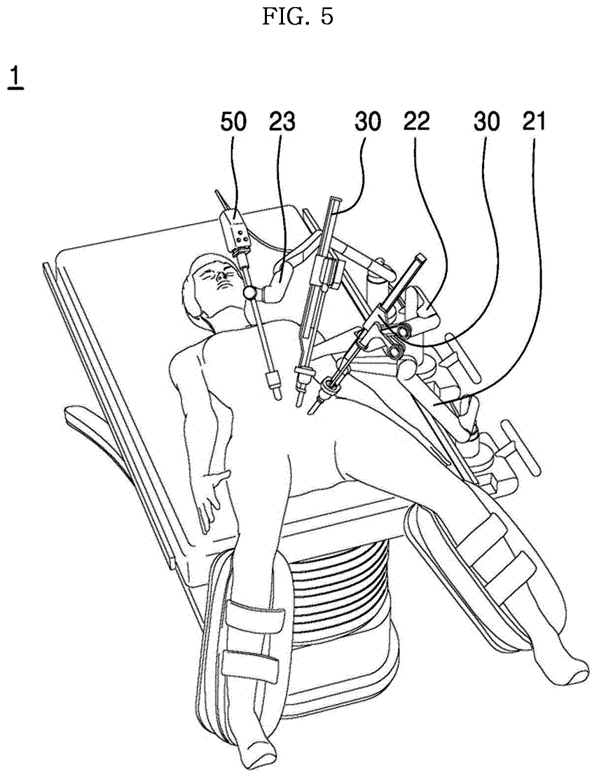

FIG. 3 is a diagram for describing another example of the system for driving a surgical instrument according to an embodiment, FIG. 4 is a block diagram illustrating an internal configuration of the surgical robot system of FIG. 3, and FIG. 5 is a perspective view illustrating a slave robot of the surgical robot system of FIG. 3 and a multi-joint type surgical instrument mounted on the slave robot.

Referring to FIGS. 3 to 5, a surgical robot system 1 includes a master robot 10, a slave robot 20, and a multi-joint type surgical instrument 30.

The master robot 10 includes manipulating members 10a and a display member 10b, and the slave robot 20 includes one or more robot arm units 21, 22, and 23.

The master robot 10 includes the manipulating members 10a so that a surgical operator can grip and manipulate them respectively with both hands. The manipulating members 10a may be implemented as two or more handles as illustrated in FIG. 3, and manipulation signals according to the handle manipulation of the user, i.e., a surgical operator, are transmitted to the slave robot 20 through a wired or wireless communication network so that the robot arm units 21, 22, and 23 are controlled. That is, surgical motions such as positioning, rotation, and cutting operations of the robot arm units 21, 22, and 23 may be performed by the handle manipulation of the surgical operator. Here, the manipulation signals may include one or more of manipulation information related to a user's motion, and first driving information or second driving information related to a motion of the surgical instrument, which are described above.

For example, the surgical operator may manipulate the robot arm units 21, 22, and 23 using manipulation levers in the form of a handle. The manipulation lever as described above may have various mechanical configurations according to the manipulate method thereof, and may be provided in various configurations for operating the robot arm units 21, 22, and 23 of the slave robot 20 and/or other surgical instruments, such as a master handle manipulating the motion of each of the robot arm units 21, 22, and 23 and various input tools added to the master robot 10 for manipulating the functions of the entire system such as joystick, keypad, trackball, foot pedal, and touch screen. Here, the manipulating member 10a is not limited to the shape of a handle and can be applied without any limitation as long as it can control motions of the robot arm units 21, 22, and 23 through a network such as a wired or wireless communication network.

Meanwhile, according to an embodiment of the present disclosure, the manipulation information may be generated based on the manipulation lever or the manipulating member 10a described above. For example, according to an embodiment of the present disclosure, the manipulation information may be generated based on a motion of a user who manipulates the manipulation lever or the manipulating member 10a. However, examples of generating the manipulation information are not limited to the above description.

Alternatively, a voice input, a motion input, or the like may also be applied to the surgical robot system 1 for user input. That is, a user may wear, on the head thereof, glasses or a head mount display (HMD), to which a sensor is attached, and a laparoscope 50 may move according to a direction according to the user's gaze. Alternatively, when the user issues a command with voice, such as “left,” “right,” “first arm,” “second arm,” and the like, the voice command may be recognized and the motion may be performed. For example, in the present disclosure, according to an embodiment, manipulation information may be generated based on a user's voice, first driving information may be calculated based on the manipulation information, the presence of a risk associated with a motion of the surgical instrument may be determined based on the first driving information, and the surgical instrument may be driven based on the result of determining the presence of the risk.

An image captured through the laparoscope 50 may be displayed as a screen image on the display member 10b of the master robot 10. In addition, a predetermined virtual manipulation plate may be displayed independently or displayed together with the image captured by the laparoscope 50 on the display member 10b.

The display member 10b may include one or more monitors, each of which may individually display information necessary for surgery. The quantity of monitors may be variously determined depending on the type or kind of information that needs to be displayed.

Meanwhile, the slave robot 20 may include one or more robot arm units 21, 22, and 23. Here, each of the robot arm units 21, 22, and 23 may be provided in the form of a module that can operate independently of each other, and in this case, an algorithm for preventing a collision between the robot arm units 21, 22, and 23 may be applied to the surgical robot system 1.

In general, a robot arm refers to a device having a function similar to that of the arm and/or the wrist of a human being and having a wrist portion to which a predetermined tool may be attached. In the present disclosure, the robot arm units 21, 22, and 23 may each be defined as a concept encompassing all of the components such as an upper arm, a lower arm, a wrist, and an elbow, a multi-joint type surgical instrument coupled to the wrist portion, and the like. Alternatively, the robot arm unit may also be defined as a concept that includes only components for driving the multi-joint type surgical instrument, excluding the multi-joint type surgical instrument coupled to the wrist portion.

The robot arm units 21, 22, and 23 of the slave robot 20 described above may be implemented to be driven with multiple degrees of freedom. The robot arm units 21, 22, and 23 may include, for example, a surgical instrument inserted into a surgical site of a patient, a yaw driving part for rotating the surgical instrument in a yaw direction according to a surgical position, a pitch driving part for rotating the surgical instrument in a pitch direction perpendicular to a rotational driving of the yaw driving part, a transfer driving part for moving the surgical instrument in a length direction, a rotation driving part for rotating the surgical instrument, and a surgical instrument driving part for incising or cutting the surgical lesion by driving an end effector at an end of the surgical instrument. However, the configuration of the robot arm units 21, 22, and 23 is not limited thereto, and it should be understood that this example does not limit the scope of the present disclosure. Here, a detailed description of the actual control process, such as rotation and movement of the robot arm units 21, 22, and 23 in a corresponding direction by the surgical operator manipulating the manipulating member 10a will be omitted.

In the exemplary embodiment shown in FIG. 5, two of the robot arm units 21, 22, and 23 may each have the multi-joint type surgical instrument 30 attached thereto, and one of the robot arm units 21, 22, and 23 may have the laparoscope 50 attached thereto. In addition, the surgical operator may select the robot arm unit 21, 22, and/or 23 to be controlled via the master robot 10. As described above, by directly controlling a total of three or more surgical instruments through the master robot 10, the surgical operator may accurately and freely control various tools according to the intention of the surgical operator without a surgical assistant.

Meanwhile, one or more slave robots 20 may be provided to operate the patient, and the laparoscope 50 for allowing a surgical site to be displayed as a screen image through the display member 10b may be implemented as an independent slave robot 20. In addition, as described above, the embodiments of the present disclosure can be used universally for surgeries in which various surgical endoscopes other than laparoscopes (e.g., thoracoscopic, arthroscopic, rhinoscopic, and the like) are used.

Meanwhile, the master robot 10 may generate manipulation information related to a user's motion for driving the surgical instrument, calculate first driving information based on the manipulation information, determine the presence of a risk associated with a motion of the surgical instrument based on the first driving information, and drive the surgical instrument based on the result of determining the presence of the risk. In addition, the master robot 10 may update the first driving information based on the result of determining the presence of the risk.

For example, the master robot 10 may transmit the first driving information to the slave robot 20 via a wired or wireless communication network to control the robot arm units 21, 22, and 23. That is, surgical motions such as positioning, rotation, and cutting operations of the robot arm units 21, 22, and 23 may be performed by the handle manipulation of the surgical operator.

Referring to FIG. 4, in an embodiment of the present disclosure, the master robot 10 may include an image input part 11, a screen display part 12, a user input part 13, a manipulation signal generation part 14, a control part 15, a memory 16, a storage part 17, and a communication part 18.

Meanwhile, the master robot 10 may be included in the user terminal of FIG. 2A. For example, the manipulation signal generation part 14, the control part 15, and the like are included in the processor 2011, the memory 16, the storage part 17, and the like are included in the memory 2012, and the communication part 18 may be included in the communication module 2014, but the example of the master robot 10 is not limited to the above description.

The image input part 11 may receive an image captured by a camera provided in the laparoscope 50 of the slave robot 20 through a wired or wireless communication network. The image captured by the camera may include an image representing the motion of the surgical instrument driven by using the first driving information or the second driving information.

The screen display part 12 outputs a screen image corresponding to the image received through the image input part 11 as visual information. In addition, the screen display part 12 may further output information corresponding to biometric information of a subject to be treated, when the biometric information is input. In addition, the screen display part 12 may further output image data (e.g., an X-ray image, a computerized tomography (CT) image, a magnetic resonance imaging (MRI) image, or the like) associated with a patient for a surgical site. Here, the screen display part 12 may be implemented in the form of a display member (see 10b of FIG. 3), and an image processing process for allowing the received image to be output as a screen image through the screen display part 12 may be performed by the control part 15. Here, the image may include an image representing the motion of the surgical instrument driven by using the first driving information or the second driving information.

In the embodiment illustrated in FIG. 4, the image input part and the screen display part are illustrated as being included in the master robot 10, but the present disclosure is not limited thereto. The display member may be provided as a separate member spaced apart from the master robot 10. Alternatively, the display member may be provided as one component of the master robot 10. In addition, in another embodiment, a plurality of display members may be provided, one of which may be disposed adjacent to the master robot 10, and the others thereof may be disposed at some distance from the master robot 10.

Here, the screen display part 12 (that is, the display member 10b of FIG. 3) may be provided as a three-dimensional display device. Specifically, the three-dimensional display device refers to an image display device in which depth information is added to a two-dimensional image by applying a stereoscopic technique, and this depth information is used to enable an observer to feel a live three-dimensional feeling and a sense of reality. The surgical robot system 1 according to an embodiment of the present disclosure may provide a more realistic virtual environment to a user by including a three-dimensional display device as the screen display part 12.

The user input part 13 is a member for allowing the surgical operator to manipulate the positions and functions of the robot arm units 21, 22, and 23 of the slave robot 20. The user input part 13 may be provided in the form of a handle-shaped manipulation member (see 10a of FIG. 3) as illustrated in FIG. 3, but the shape thereof is not limited thereto and may be implemented by being modified in various shapes to achieve the same purpose. In addition, for example, a portion of the user input part 13 may be provided in the form of a handle, and the other portions thereof may be provided in different forms, such as a clutch button. In addition, a finger insertion tube or insertion ring may be further formed so as to allow the surgical operator's finger to be inserted therethrough and fixed to facilitate manipulation of the surgical tool.

Meanwhile, according to an embodiment of the present disclosure, the manipulation information may be generated based on a motion of the surgical operator with respect to the user input part 13. For example, according to an embodiment of the present disclosure, the manipulation information may be generated based on a motion of the surgical operator who manipulates the user input part 13. However, the example of generating manipulation information is not limited to the above description.

When the surgical operator manipulates the user input part 13 to control positional movements or surgical motions of the robot arm units 21, 22, and 23, the manipulation signal generation part 14 may generate a manipulation signal corresponding thereto. In an example, when the surgical operator manipulates the user input part 13 to control the positional movements or surgical motions of the robot arm units 21, 22, and 23, the manipulation signal generation part 14 may generate manipulation information corresponding thereto.

For example, the manipulation signal generation part 14 transmits the generated manipulation signal to the control part 15 or transmits the generated manipulation signal to the slave robot 20 through the communication part 18. The manipulation signal may be transmitted and received via a wired or wireless communication network. Based on the transmitted manipulation signal, the control part 15 may control the slave robot 20 or the multi-joint type surgical instrument 30 to operate. Alternatively, based on the transmitted manipulation signal, the robot arm control part 26 included in the slave robot 20 may control the robot arm units 21, 22, and 23 to operate. Alternatively, based on the transmitted manipulation signal, the instrument control part 27 included in the slave robot 20 may control the multi-joint type surgical instrument 30 to operate. However, the method by which the motion of the slave robot 20 or the multi-joint type surgical instrument 30 is controlled based on the manipulation signal is not limited to the above description.

The instrument control part 27 may receive a manipulation signal generated by the manipulation signal generation part 14 of the master robot 10, and may serve to control the multi-joint type surgical instrument 30 so as to operate according to the manipulation signal.

The control part 15 is may be a central processing device that controls operations of each component so that the above-described functions can be performed. In an example, the control part 15 may perform a function of converting an image input through the image input part 11 into a screen image to be displayed through the screen display part 12. In another example, the control part 15 may generate first driving information related to positional movements or surgical motions of the robot arm units 21, 22, and 23 based on the manipulation information. In addition, the control part 15 may determine the presence of a risk associated with the positional movements or the surgical motions of the robot arm units 21, 22, and 23 based on the first driving information. In addition, the control part 15 may update the first driving information based on the result of determining the presence of the risk. In addition, the control part 15 may drive the robot arm units 21, 22, and 23 based on the result of determining the presence of the risk. In addition, the control part 15 may calculate second driving information based on the first driving information, and may drive the robot arm units 21, 22, and 23 based on the second driving information.

Meanwhile, according to the above description, it has been described that the control part 15 calculates the first driving information, updates the first driving information, or calculates the second driving information based on the first driving information, but the present disclosure is not limited thereto, and other control parts (e.g., the robot arm control part 26, the instrument control part 27, and the like) according to the present disclosure may perform those operations.

The memory 16 may perform a function of temporarily or permanently storing data processed by the control part 15. Here, the memory 16 may include a magnetic storage medium or a flash storage medium, but the scope of the present disclosure is not limited thereto.

The storage part 17 may store data received from the slave robot 20. In addition, the storage part 17 may store various pieces of input data (e.g., patient data, device data, surgery data, and the like).

The communication part 18 interworks with a communication network 60 to provide a communication interface necessary for transmitting and receiving image data transmitted from the slave robot 20 and control data transmitted from the master robot 10. The image data transmitted from the slave robot 20 may include an image representing a motion of the surgical instrument driven by using the first driving information or the second driving information. The control data transmitted from the master robot 10 may include the first driving information or the second driving information related to the motion of the slave robot 20.

According to the embodiment shown in FIG. 4, the slave robot 20 may include a plurality of motion assist robot unit control parts 21a, 22a, and 23a. In addition, the first motion assist robot unit control part 21a includes the robot arm control part 26, the instrument control part 27, and a communication part 29. In addition, the first motion assist robot unit control part 21a may further include a rail control part 28.

The robot arm control part 26 may receive a manipulation signal generated by the manipulation signal generation part 14 of the master robot 10, and may serve to control the robot arm units 21, 22, and 23 so as to operate according to the manipulation signal. For example, the robot arm control part 26 may serve to receive first driving information or second driving information calculated (or updated) by the master robot 10, and control the robot arm units 21, 22, and 23 to operate according to the first driving information or the second driving information.

The instrument control part 27 may receive a manipulation signal generated by the manipulation signal generation part 14 of the master robot 10, and may serve to control the multi-joint type surgical instrument 30 so as to operate according to the manipulation signal. For example, the instrument control part 27 may serve to receive first driving information calculated (or updated) by the master robot 10 and control the multi-joint type surgical instrument 30 to operate according to the first driving information.

The communication part 29 interworks with the communication network 60 to provide a communication interface necessary for transmitting and receiving image data transmitted from the slave robot 20 and control data transmitted from the master robot 10. The image data transmitted from the slave robot 20 may include an image representing a motion of the surgical instrument driven by using the first driving information or the second driving information. The control data transmitted from the master robot 10 may include first driving information or second driving information related to a motion of the slave robot 20.

Meanwhile, the communication network 60 serves to connect the master robot 10 and the slave robot 20. That is, the communication network 60 refers to a communication network for providing an access path so that data can be transmitted and received between the master robot 10 and the slave robot 20 after the master robot 10 and the slave robot 20 are connected. The communication network 60 may be, for example, a wired network such as local area networks (LANs), wired area networks (WANs), metropolitan area networks (MANs), and integrated service digital networks (ISDNs), or a wireless network such as wireless LANs, code division multiple access (CDMA), Bluetooth™, and satellite communication, but the scope of the present disclosure is not limited thereto.

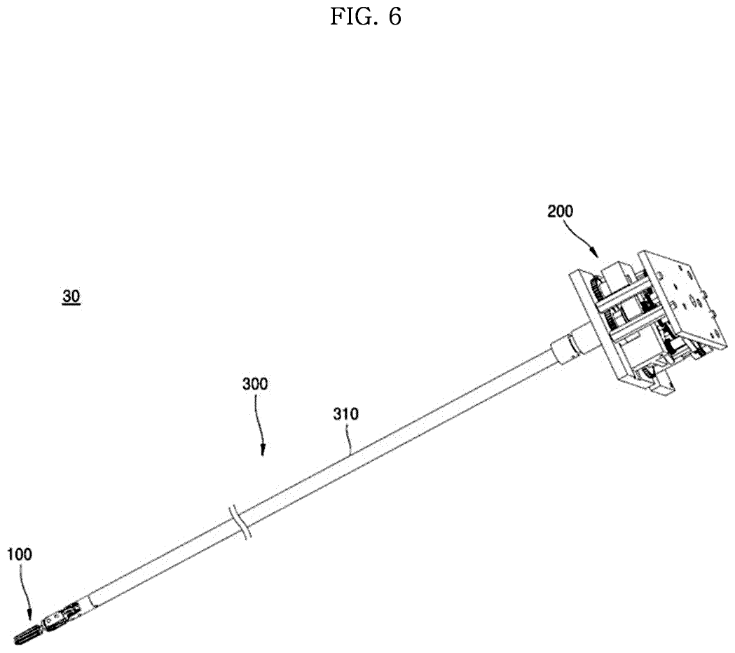

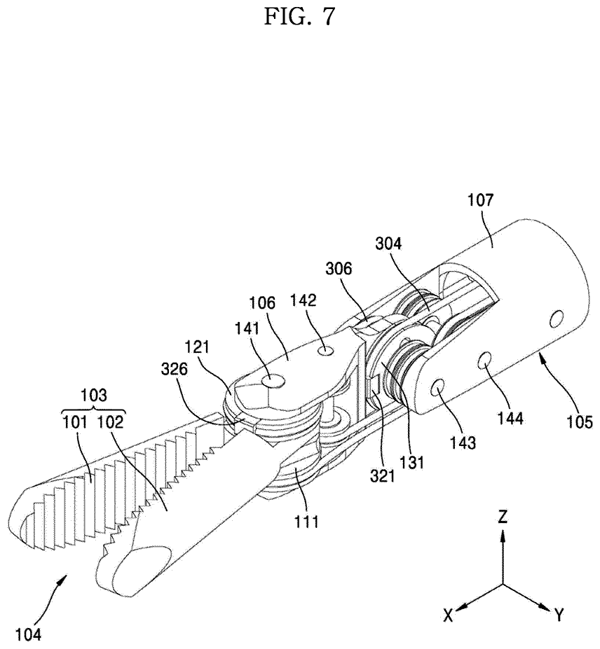

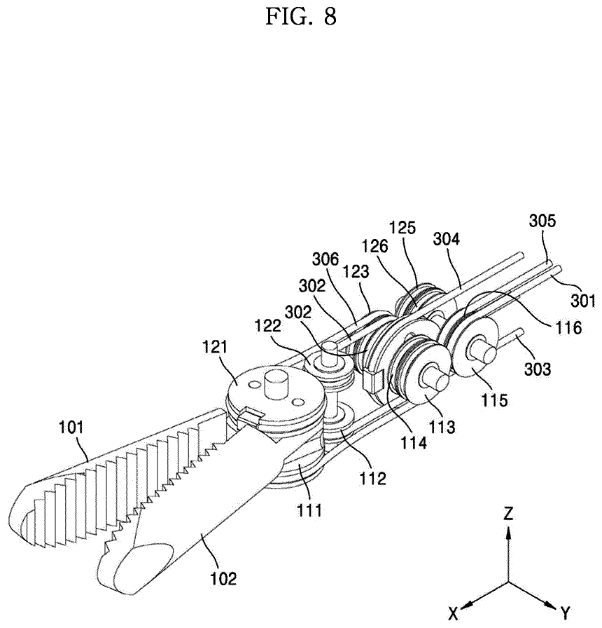

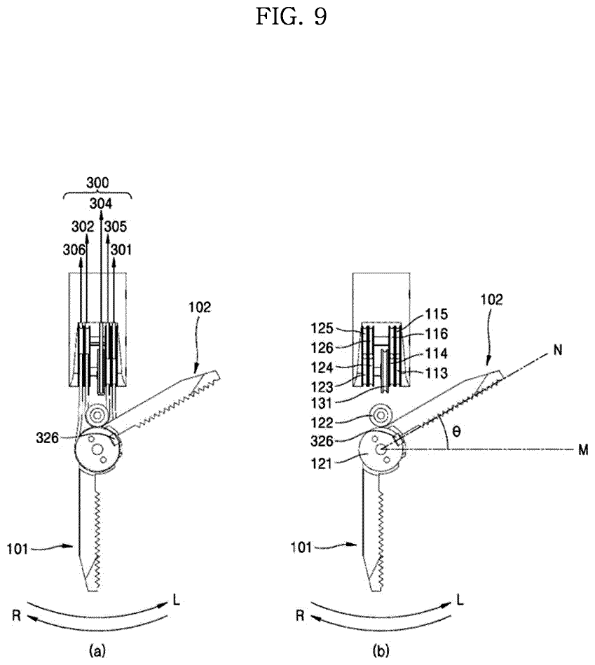

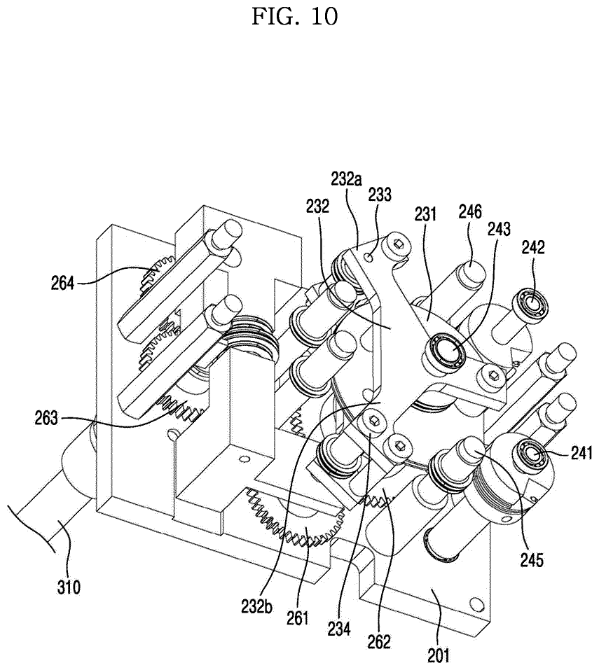

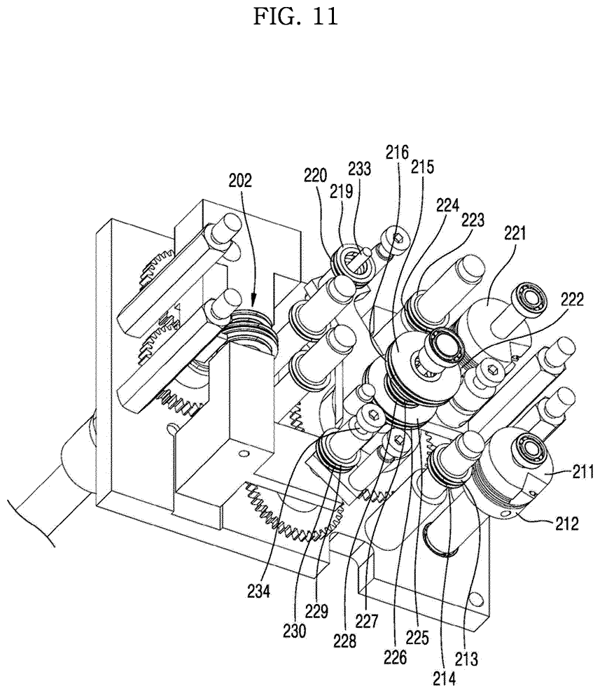

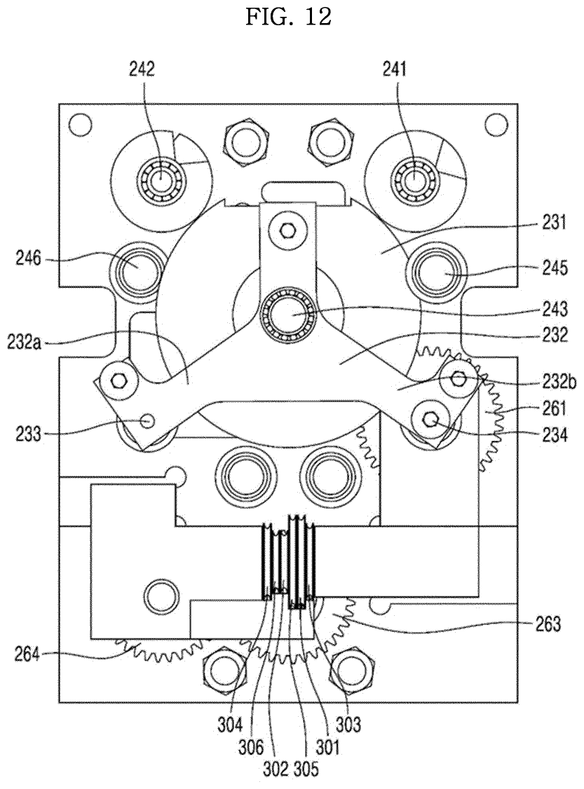

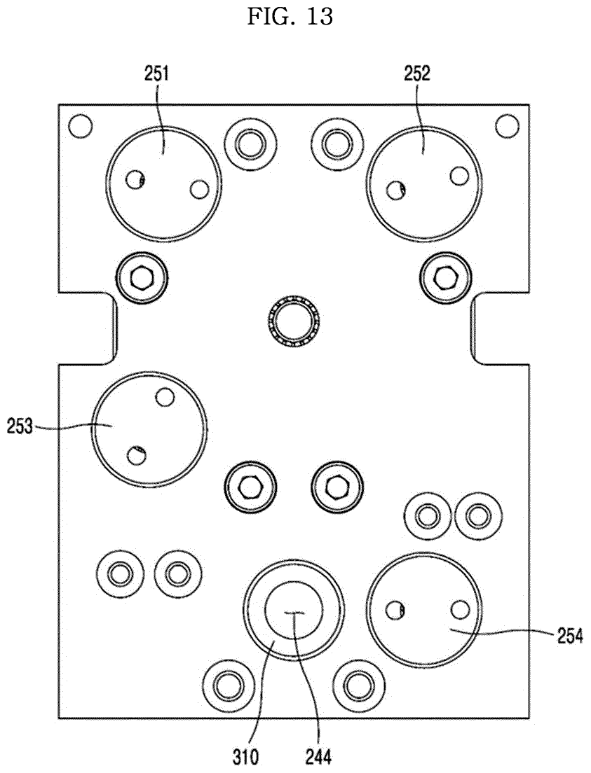

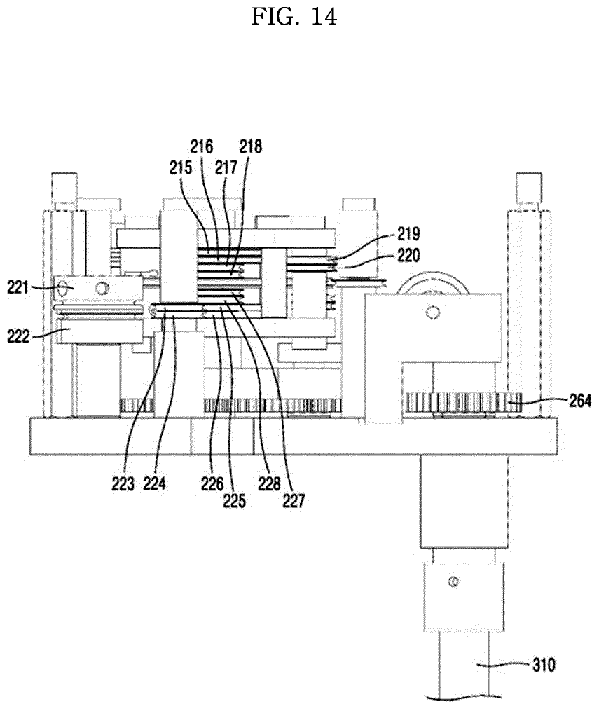

FIG. 6 is a perspective view illustrating a multi-joint type surgical instrument according to an embodiment of the present disclosure, FIGS. 7 and 8 are perspective views of an end tool of the multi-joint type surgical instrument of FIG. 6, and FIG. 9 is a plan view of the end tool of the multi-joint type surgical instrument of FIG. 6. FIGS. 10 and 11 are perspective views of a driving part of the multi-joint type surgical instrument of FIG. 6, FIG. 12 is a plan view of the driving part of the multi-joint type surgical instrument of FIG. 6, FIG. 13 is a rear view of the driving part of the multi-joint type surgical instrument of FIG. 6, and FIG. 14 is a side view of the driving part of the multi-joint type surgical instrument of FIG. 6.

Referring first to FIG. 6, the multi-joint type surgical instrument 30 according to an embodiment of the present disclosure may include an end tool 100, a driving part 200, and a power transmission part 300, and the power transmission part 300 may include a connection part 310.

The connection part 310 is formed in the shape of a hollow shaft, in which one or more wires (to be described later) may be accommodated, and may have one end portion to which the driving part 200 is coupled and the other end portion to which the end tool 100 is coupled, and serve to connect the driving part 200 and the end tool 100.

The driving part 200 is formed at one end portion of the connection part 310 and provides an interface capable of being coupled to the robot arm unit (see 21 or the like in FIG. 3). Accordingly, when a user operates the master robot (see 10 in FIG. 3), a motor (not shown) of the robot arm unit (see 21 or the like in FIG. 3) is operated so that the end tool 100 of the multi-joint type surgical instrument 30 can perform a motion corresponding thereto, and a driving force of the motor (not shown) is transmitted to the end tool 100 through the driving part 200. In other words, it may be described that the driving part 200 itself becomes an interface that connects between the multi-joint type surgical instrument 30 and the slave robot 20.

For example, when the user input part 13 (see FIG. 3) is operated by a user, a motor (not shown) of the robot arm unit 21 or the like (see FIG. 3) operates so that the end tool 100 of the multi-joint type surgical instrument 30 can perform a motion corresponding thereto, and a driving force of the motor (not shown) may be transmitted to the end tool 100 through the driving part 200.

The end tool 100 is formed on the other end portion of the connection part 310, and performs necessary motions for surgery by being inserted into a surgical site. In an example of the above-described end tool 100, as shown in FIG. 7, a pair of jaws 101 and 102 for performing a grip motion may be used. However, the embodiment of the present disclosure is not limited thereto, and various devices for performing surgery may be used as the end tool 100. For example, a configuration such as a cantilever cautery may also be used as the end tool. The above-described end tool 100 is connected to the driving part 200 by the power transmission part 300 and receives a driving force through the power transmission part 300 to perform a motion necessary for surgery, such as a gripping motion, a cutting motion, a suturing motion, or the like.

Here, the end tool 100 of the multi-joint type surgical instrument 30 according to an embodiment of the present disclosure is formed to be rotatable in at least two or more directions, for example, the end tool 100 may be formed to perform a pitch motion around a rotation shaft 143 of FIG. 7 and simultaneously perform a yaw motion and an actuation motion around a rotation shaft 141 of FIG. 7.

Here, each of a pitch motion, a yaw motion, an actuation motion, and a roll motion as used in the present disclosure are defined as follows.

First, the pitch motion means a motion of the end tool 100 rotating in a vertical direction with respect to an extension direction of the connection part 310 (an X-axis direction of FIG. 6), that is, a motion rotating around the Y-axis of FIG. 7. In other words, the pitch motion means a motion of the end tool 100, which is formed to extend from the connection part 310 in the extension direction of the connection part 310 (the X-axis direction of FIG. 7), rotating vertically around the Y-axis with respect to the connection part 310.

Next, the yaw motion means a motion of the end tool 100 rotating in left and right directions, that is, a motion rotating around a Z-axis of FIG. 7, with respect to the extension direction of the connection part 310 (the X-axis direction of FIG. 7). In other words, the yaw motion means a motion of the end tool 100, which is formed to extend from the connection part 310 in the extension direction of the connection part 310 (the X-axis direction of FIG. 7), rotating horizontally around the Z-axis with respect to the connection part 310. That is, the yaw motion relates to a motion of two jaws 101 and 102, which are formed on the end tool 100, rotating around the Z-axis in the same direction.

Meanwhile, the actuation motion means a motion of the end tool 100 rotating around the same shaft of rotation as that of the yaw motion, while the two jaws 101 and 102 rotate in the opposite directions so as to be closed or opened. That is, the actuation motion means rotating motions of the two jaws 101 and 102, which are formed on the end tool 100, in the opposite directions around the Z-axis.

Defining this from another perspective, the yaw rotation may be defined as a motion in which an end tool jaw pulley (to be described later) rotates around the rotation shaft 141, which is an end tool jaw pulley rotation shaft, and the pitch rotation may be defined as a motion in which the end tool jaw pulley revolves around the rotation shaft 143, which is an end tool pitch rotation shaft.

The roll motion refers to a motion in which the multi-joint type surgical instrument rotates with the connection part 310 as a shaft. For example, the roll motion may be a motion in which the multi-joint type surgical instrument rotates in the clockwise or counterclockwise direction around the extension direction of the connection part 310 (the X-axis direction of FIG. 7).

Meanwhile, the roll motion may mean a motion in which the end tool 100 rotates around the X-axis with respect to the connection part 310. For example, the roll motion may be a motion in which the end tool rotates in the clockwise or counterclockwise direction around the extension direction of the connection part 310 (the X-axis direction of FIG. 7).

The power transmission part 300 may connect the driving part 200 and the end tool 100, transmit the driving force from the driving part 200 to the end tool 100, and include a plurality of wires, pulleys, links, sections, gears, or the like.

Hereinafter, the end tool 100, the driving part 200, the power transmission part 300, and the like of the multi-joint type surgical instrument 30 of FIG. 6 will be described in more detail.

Hereinafter, the power transmission part 300 of the multi-joint type surgical instrument 30 of FIG. 6 will be described in more detail.

Referring to FIGS. 6 to 14, the power transmission part 300 of the multi-joint type surgical instrument 30 according to an embodiment of the present disclosure may include a plurality of wires 301, 302,303, 304, 305, and 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In addition, the wires 303 and 304 may be paired to serve as pitch wires.

Here, in the drawings, a pair of wires are illustrated as being associated with a rotational motion of a first jaw 101, and a pair of wires are illustrated as being associated with a rotational motion of a second jaw 102, but an embodiment of the present disclosure is not limited thereto. For example, a pair of wires may be associated with a yaw motion, and a pair of wires may be associated with an actuation motion.

In addition, the power transmission part 300 of the multi-joint type surgical instrument 30 according to an embodiment of the present disclosure may include a coupling member 321, a coupling member 326, and the like, which are coupled to respective end portions of the wires in order to couple the wires and the pulleys. Here, each of the coupling members may have various shapes as necessary, such as a ball shape, a tube shape, and the like.

Here, the coupling member 321, which is a pitch wire coupling member, is coupled to the end portions of the wires 303 and 304, which are pitch wires, at the end tool 100 side to serve as a pitch wire-end tool coupling member. Meanwhile, although not illustrated in the drawings, a pitch wire-driving part coupling member (not shown) may be coupled to the end portions of the wires 303 and 304, which are pitch wires, at the driving part 200 side.

Meanwhile, the coupling member 326, which is a second jaw wire coupling member, is coupled to the end portions of the wires 302 and 306, which are second jaw wires, at the end tool 100 side to serve as a second jaw wire-end tool coupling member. Meanwhile, although not illustrated in the drawings, a second jaw wire-driving part coupling member (not shown) may be coupled to the end portions of the wires 302 and 306, which are second jaw wires, at the driving part 200 side.

Meanwhile, although not illustrated in the drawings, a coupling member (not shown) having the same shape as the second jaw wire coupling member 326 may be coupled to the end portions of the wires 301 and 305, which are first jaw wires, at the end tool 100 side to serve as a first jaw wire-end tool coupling member. Meanwhile, although not illustrated in the drawings, a first jaw wire-driving part coupling member (not shown) may be coupled to the end portions of the wires 301 and 305, which are first jaw wires, at the driving part 200 side.

Here, each of the coupling members is classified as being included in the power transmission part 300, but the coupling members may be classified such that the coupling member at the end tool 100 side may be included in the end tool 100, and the coupling member at the driving part 200 side may be included in the driving part 200.

The coupling relationship between the wires, the fastening members, and the respective pulley will be described in detail as follows.

First, the wires 302 and 306, which are second jaw wires, may be a single wire. The second jaw wire coupling member 326, which is a second jaw wire-end tool coupling member, is inserted at an intermediate point of the second jaw wire, which is a single wire, and the second jaw wire coupling member 326 is crimped and fixed, and then, both strands of the second jaw wire centered on the second jaw wire coupling member 326 may be referred to as the wire 302 and the wire 306, respectively.

Alternatively, the wires 302 and 306, which are second jaw wires, may also be formed as separate wires, and connected to each other by the second jaw wire coupling member 326.

In addition, by coupling the second jaw wire coupling member 326 to a pulley 121, the wires 302 and 306 may be fixedly coupled to the pulley 121. This allows the pulley 121 to rotate as the wires 302 and 306 are pulled and released.

Meanwhile, the second jaw wire-driving part coupling member (not shown) may be coupled to the end portions of the wires 302 and 306, which are opposite to the end portions to which the second jaw wire coupling member 326 is coupled. That is, the second jaw wire-driving part coupling member (not shown) may be fixed to each of the wires 302 and 306 by inserting the opposite end portions of the wires 302 and 306 into the second jaw wire-driving part coupling member (not shown) and crimping the coupling member (not shown).

In addition, by coupling the second jaw wire-driving part coupling member (not shown) coupled to the wires 302 and 306 to each of the pulley 221 and the pulley 222, the wire 302 and the wire 306 may be fixedly coupled to the pulley 221 and the pulley 222, respectively. As a result, when the pulley 221 and the pulley 222 are rotated by a motor or a human force, the pulley 121 of the end tool 100 may be rotated as the wire 302 and the wire 306 are pulled and released.

Here, a driving part second jaw pulley may include two pulleys of the pulley 221 and the pulley 222, and thus the second jaw wire-driving part coupling member may also include two coupling members. Alternatively, the driving part second jaw pulley includes one pulley, the second jaw wire-driving part coupling member also includes one coupling member, and the wires 302 and 306 may be coupled to one coupling member to be coupled to one driving part second jaw pulley.

In the same manner, the wire 301 and the wire 305, which are first jaw wires, are coupled to the first jaw wire-end tool coupling member (not shown) and the first jaw wire-driving part coupling member (not shown), respectively. In addition, the first jaw wire-end tool coupling member (not shown) is coupled to a pulley 111, and the first jaw wire-driving part coupling member (not shown) is coupled to a pulley 211 and a pulley 212. As a result, when the pulleys 211 and 212 are rotated by a motor or a human force, the pulley 111 of the end tool 100 may be rotated as the wire 301 and the wire 305 are pulled and released.

In the same manner, each of one end portions of the wires 303 and 304, which are pitch wires, is coupled to the pitch wire coupling member 321, which is a pitch wire-end tool coupling member, and each of the other end portions of the wires 303 and 304 are coupled to the pitch wire-driving part coupling member (not shown). In addition, the pitch wire coupling member 321 is coupled to a pulley 131, and the pitch wire-driving part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to a coupling member 323, which is a first jaw wire-end tool coupling member, and the first jaw wire-driving part coupling member (not shown) so as to form as a whole a closed loop. Similarly, the second jaw wire and the pitch wire may each be formed to form a closed loop.

Hereinafter, the end tool 100 of the multi-joint type surgical instrument 30 of FIG. 6 will be described in more detail.

FIGS. 7 and 8 are perspective views illustrating the end tool of the multi-joint type surgical instrument of FIG. 6, and FIG. 7 is a plan view illustrating the end tool of the multi-joint type surgical instrument of FIG. 6. Here, FIG. 7 illustrates a state in which an end tool hub 106 and a pitch hub 107 are coupled, and FIG. 8 illustrates a state in which the end tool hub 106 and the pitch hub 107 are removed.

Referring to FIGS. 7 to 9, the end tool 100 according to an embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as a jaw 103.

Further, the end tool 100 may include the pulley 111, a pulley 112, a pulley 113, a pulley 114, a pulley 115, and a pulley 116 that are related to a rotational motion of the first jaw 101. In addition, the end tool 100 may include the pulley 121, a pulley 122, a pulley 123, a pulley 124, a pulley 125, and a pulley 126 that are related to a rotational motion of the second jaw 102.

Here, in the drawings, one group of pulleys are illustrated as being associated with a rotational motion of the first jaw 101, and one group of pulleys are illustrated as being associated with a rotational motion of the second jaw 102, but an embodiment of the present disclosure is not limited thereto. For example, one group of pulleys in the end tool may be associated with a yaw motion, and one group of pulleys in the end tool may be associated with an actuation motion. Here, the pulleys included in the end tool 100, including the pulleys described above, may be collectively referred to as end tool pulleys.

Meanwhile, the pulleys facing each other are illustrated in the drawings as being formed parallel to each other, but an embodiment of the present disclosure is not limited thereto, and each of the pulleys may be variously formed with a position and a size suitable for the configuration of the end tool.

Further, the end tool 100 according to an embodiment of the present disclosure may include the end tool hub 106 and the pitch hub 107.

The rotation shaft 141 and a rotation shaft 142, which will be described later, may be inserted through the end tool hub 106, and the end tool hub 106 may internally accommodate at least some of the first jaw 101 and the second jaw 102, which are axially coupled to the rotation shaft 141. In addition, the end tool hub 106 may internally accommodate at least some of the pulley 112 and the pulley 122 that are axially coupled to the rotation shaft 142.

In addition, the pulley 131 serving as an end tool pitch pulley may be formed at one end portion of the end tool hub 106. As shown in FIG. 7, the pulley 131 may be formed as a separate member from the end tool hub 106 and coupled to the end tool hub 106. Alternatively, although not illustrated in the drawings, the pulley 131 may be integrally formed with the end tool hub 106 as one body. That is, one end portion of the end tool hub 106 is formed in a disk shape or a semi-circular shape such as a pulley, and a groove around which a wire can be wound may be formed on an outer circumferential surface thereof. The wires 303 and 304 described above are coupled to the pulley 131 serving as an end tool pitch pulley, and a pitch motion may be performed as the pulley 131 is rotated around the rotation shaft 143.

The rotation shaft 143 and a rotation shaft 144, which will be described later, may be inserted through the pitch hub 107, and the pitch hub 107 may be axially coupled to the end tool hub 106 and the pulley 131 by the rotation shaft 143. Thus, the end tool hub 106 and the pulley 131 (coupled thereto) may be formed to be rotatable around the rotation shaft 143 with respect to the pitch hub 107.

Further, the pitch hub 107 may internally accommodate at least some of the pulley 113, the pulley 114, the pulley 123, and the pulley 124 that are axially coupled to the rotation shaft 143. In addition, the pitch hub 107 may internally accommodate at least some of the pulley 115, the pulley 116, the pulley 125, and the pulley 126 that are axially coupled to the rotation shaft 144.

Further, the end tool 100 according to an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105 thereof. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed according to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as end tool jaw auxiliary pulleys.

Specifically, the pulley 112 and the pulley 122, which are end tool jaw auxiliary pulleys, may be additionally provided on one side of the pulley 111 and one side of the pulley 121, respectively. In other words, the pulley 112, which is an auxiliary pulley, may be disposed between the pulley 111 and the pulley 113/pulley 114. In addition, the pulley 122, which is an auxiliary pulley, may be disposed between the pulley 121 and the pulley 123/pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft. Such auxiliary pulleys will be described in more detail later.

The pulley 113 and the pulley 114 function as end tool first jaw pitch main pulleys, and the pulley 123 and the pulley 124 function as end tool second jaw pitch main pulleys, and these two components may be collectively referred to as end tool jaw pitch main pulleys.

The pulley 115 and the pulley 116 function as end tool first jaw pitch sub-pulleys, and the pulley 125 and the pulley 126 function as end tool second jaw pitch sub-pulleys, and these two components may be collectively referred to as end tool jaw pitch sub-pulleys.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In addition, the pulley 115 and the pulley 116 are disposed on one side of the pulley 113 and on one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but an embodiment of the present disclosure is not limited thereto, and the rotation axes of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the first jaw wire-end tool coupling member 323 is sequentially wound to make contact with at least portions of the pulley 111, the pulley 112, the pulley 114, and the pulley 116 in turn.

Viewed from another perspective, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow 301 of FIG. 9, a coupling member (not shown) to which the wire 301 is coupled and the pulley 111 coupled to the coupling member (not shown) are rotated in an arrow L direction of FIG. 9. In contrast, when the wire 305 is pulled in the direction of an arrow 305 of FIG. 9, a coupling member (not shown) to which the wire 305 is coupled and the pulley 111 coupled to the coupling member (not shown) are rotated in an arrow R direction of FIG. 9.

Hereinafter, the pulley 112 and the pulley 122 serving as auxiliary pulleys will be described in more detail.

The pulley 112 and the pulley 122 may serve to increase rotation angles of the first jaw 101 and the second jaw 102, respectively, by coming into contact with the wire 305, which is a first jaw wire, and the wire 302, which is a second jaw wire, and changing the arrangement paths of the wires 305 and 302 to a certain extent.

That is, when the auxiliary pulleys are not disposed, each of the first jaw and the second jaw may be rotated up to a right angle, but in an embodiment of the present disclosure, the pulley 112 and the pulley 122, which are auxiliary pulleys, are additionally provided, so that the maximum rotation angle may be increased by θ as shown in FIG. 9. This enables a motion of the two jaws of the end tool 100 being opened for an actuation motion while the two jaws are yaw-rotated by 90° in the L direction. This is because the second jaw 102 is rotated by the additional angle θ as shown in FIG. 9. Similarly, an actuation motion is possible even when the two jaws are yaw-rotated in the R direction. In other words, a feature of increasing the range of yaw rotation in which an actuation motion is possible may be obtained through the pulley 112 and the pulley 122.

This will be described in more detail as follows.

When the auxiliary pulleys are not disposed, since the first jaw wire is fixedly coupled to the end tool first jaw pulley, and the second jaw wire is fixedly coupled to the end tool second jaw pulley, each of the end tool first jaw pulley and the end tool second jaw pulley may be rotated up to 90°. In this case, when the actuation motion is performed while the first jaw and the second jaw are located at a 90° line, the first jaw may be opened, but the second jaw may not be rotated beyond 90°. Accordingly, when the first jaw and the second jaw perform a yaw motion over a certain angle, there was a problem that the actuation motion is not smoothly performed.

In order to address such a problem, in the multi-joint type surgical instrument 30 according to an embodiment of the present disclosure, the pulley 112 and the pulley 122, which are auxiliary pulleys, are additionally disposed at one side of the pulley 111 and one side of the pulley 121, respectively. As described above, as the arrangement paths of the wire 305, which is a first jaw wire, and the wire 302, which is a second jaw wire, are changed to a certain extent by disposing the pulley 112 and the pulley 122, a tangential direction of the wires 305 and 302 is changed, and accordingly, the second jaw wire coupling member 326 for coupling the wire 302 and the pulley 121 may be rotated up to a line N of FIG. 9. That is, the second jaw wire coupling member 326, which is a coupling part of the wire 302 and the pulley 121, is rotatable until the second jaw wire coupling member 326 is located on a common internal tangent of the pulley 121 and the pulley 122. Similarly, the first jaw wire-end tool coupling member 323, which is a coupling part of the wire 305 and the pulley 111, is rotatable until the first jaw wire-end tool coupling member 323 is located on a common internal tangent of the pulley 111 and the pulley 112, so that the range of rotation in the L direction may be increased.

In other words, by the pulley 112, the wires 301 and 305, which are two strands of the first jaw wire wound around the pulley 111, are disposed at one side with respect to a plane perpendicular to the Y-axis and passing through the X-axis. Simultaneously, by the pulley 122, the wires 302 and 306, which are two strands of the second jaw wire wound around the pulley 121, are disposed at the other side with respect to the plane perpendicular to the Y-axis and passing through the X-axis.

In other words, the pulley 113 and the pulley 114 are disposed at one side with respect to the plane perpendicular to the Y-axis and passing through the X-axis, and the pulley 123 and the pulley 124 are disposed at the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According the above-described embodiment of the present disclosure, as the rotation radii of the jaw 101 and the jaw 102 increase, an effect of increasing a yaw motion range in which a normal opening/closing actuation motion is performed may be obtained.

Next, components related to the rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 that is an end tool pitch rotation shaft. In addition, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144, which is an end tool pitch auxiliary rotation shaft. Here, in the drawings, it is illustrated that all of the pulley 123, the pulley 125, the pulley 124, and the pulley 126 are formed to be rotatable around the Y-axis direction, but an embodiment of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the second jaw wire coupling member 326 is sequentially wound to make contact with at least portions of the pulley 121, the pulley 122, the pulley 124, and the pulley 126.

Viewed from another perspective, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow 306 of FIG. 9, the second jaw wire coupling member 326 to which the wire 306 is coupled and the pulley 121 coupled to the second jaw wire coupling member 326 are rotated in the arrow R direction of FIG. 9. In contrast, when the wire 302 is pulled in the direction of an arrow 302 of FIG. 9, the second jaw wire coupling member 326 to which the wire 302 is coupled and the pulley 121 coupled to the second jaw wire coupling member 326 are rotated in the arrow L direction of FIG. 9.

Hereinafter, a pitch motion of the present disclosure will be described in more detail.

First, for the pitch motion, at the end tool 100 side, the pulley 113, the pulley 114, the pulley 123, and the pulley 124, which are end tool jaw pitch main pulleys, are formed to be rotatable around the rotation shaft 143. Meanwhile, in a direction of the proximal end 105 of the end tool jaw pitch main pulley, the pulley 115, the pulley 116, the pulley 125, and the pulley 126, which are end tool jaw pitch sub-pulleys, are formed to be rotatable around the rotation shaft 144.

In addition, based on a plane perpendicular to the rotation shaft 141 and including the rotation shaft 143 (i.e., an XY plane), the wires 301 and 305, which are two strands of the first jaw wire, are located on the same side with respect to the XY plane. That is, the wire 301 and the wire 305 are formed to pass through lower sides of the pulley 113 and the pulley 114, which are end tool jaw pitch main pulleys, and upper sides of the pulley 115 and the pulley 116, which are end tool jaw pitch sub-pulleys.

Similarly, the wires 302 and 306, which are two strands of the second jaw wire, are located on the same side with respect to the XY plane. That is, the wires 302 and 306 are formed to pass through upper sides of the pulley 123 and the pulley 124, which are end tool jaw pitch main pulleys, and lower sides of the pulley 125 and the pulley 126, which are end tool jaw pitch sub-pulleys.

In addition, in the wires 301 and 305 that are two strands of the first jaw wire, when the wire 301 is pulled toward the arrow 301 of FIG. 9 and simultaneously the wire 305 is pulled toward the arrow 305 of FIG. 9 (i.e., when both strands of the first jaw wire are pulled in the same direction), as shown in FIG. 7, since the wires 301 and 305 are wound around lower portions of the pulleys 113 and 114, which are rotatable around the rotation shaft 143 that is an end tool pitch rotation shaft, the pulley 111 to which the wire 301 and the wire 305 are fixedly coupled, and the end tool hub 106 to which the pulley 111 is coupled are rotated together as a whole in a counterclockwise direction around the rotation shaft 143, as a result, the end tool 100 performs the pitch motion while rotating downward. At this time, since the second jaw 102 and the wires 302 and 306 fixedly coupled thereto are wound around the upper portions of the pulleys 123 and 124 rotatable around the rotation shaft 143, the wires 302 and 306 are unwound in opposite directions of the arrows 302 and 306, respectively.

In contrast, in the wires 302 and 306 that are two strands of the second jaw wire, when the wire 302 is pulled toward the arrow 302 of FIG. 9 and simultaneously the wire 306 is pulled toward the arrow 306 of FIG. 9 (i.e., when both strands of the second jaw wire are pulled in the same direction), as shown in FIG. 7, since the wires 302 and 306 are wound around lower portions of the pulleys 123 and 124, which are rotatable around the rotation shaft 143 that is an end tool pitch rotation shaft, the pulley 121 to which the wire 302 and the wire 306 are fixedly coupled, and the end tool hub 106 to which the pulley 121 is coupled are rotated together as a whole in a clockwise direction around the rotation shaft 143. As a result, the end tool 100 performs the pitch motion while rotating upward. At this time, since the first jaw 101 and the wires 301 and 305 fixedly coupled thereto are wound around the lower portions of the pulleys 113 and 114 rotatable around the rotation shaft 143, the wires 302 and 306 are moved in opposite directions of the arrows 301 and 305, respectively.

Viewed from another perspective, it may be also described that both strands of each jaw wire are moved simultaneously in the same direction when the end tool 100 is pitch-rotated.

Meanwhile, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 that are pitch wires. Specifically, the pulley 131 of the end tool 100 is rotatable around the rotation shaft 143, which is an end tool pitch rotation shaft, and may be integrally formed with the end tool hub 106 (or fixedly coupled to the end tool hub 106) as one body. In addition, the wires 303 and 304 may serve to connect the pulley 131 of the end tool 100 to the pulley 231 of the driving part 200.

Thus, when the pulley 231 of the driving part 200 is rotated, the rotation of the pulley 231 is transmitted to the pulley 131 of the end tool 100 via the wires 303 and 304, which causes the pulley 131 to also be rotated, and as a result, the end tool 100 performs a pitch motion while rotating.

That is, in the multi-joint type surgical instrument 30 according to an embodiment of the present disclosure, by providing the pulley 131 of the end tool 100, the pulley 231 of the driving part 200, and the wires 303 and 304 of the power transmission part 300 to transmit power for a pitch motion, the driving force for a pitch motion from the driving part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of a driving part relay pulley of the driving part 200, which will be described later, to a diameter of a driving part pitch pulley 231. This will be described in detail later.

Hereinafter, the driving part 200 of the multi-joint type surgical instrument 30 of FIG. 6 will be described in more detail.

Referring to FIGS. 10 to 16, the driving part 200 of the multi-joint type surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are related to a rotational motion of the first jaw 101 In addition, the driving part 200 may include the pulley 221, the pulley 222, a pulley 223, a pulley 224, a pulley 225, a pulley 226, a pulley 227, a pulley 228, a pulley 229, and a pulley 230, which are related to a rotational motion of the second jaw 102.

Here, the pulleys facing each other are illustrated in the drawings as being formed parallel to each other, but an embodiment of the present disclosure is not limited thereto, and each of the pulleys may be variously formed with a position and a size suitable for the configuration of the driving part.

In addition, the driving part 200 of the multi-joint type surgical instrument 30 according to an embodiment of the present disclosure may further include the pulley 231 serving as a driving part pitch pulley, and a pitch-yaw connector 232 configured to connect the pulley 231 to the above-described jaw pulleys of the driving part.

Further, the driving part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a first jaw rotation shaft of the driving part, and the rotation shaft 242 may function as a second jaw rotation shaft of the driving part. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft of the driving part, and the rotation shaft 246 may function as a driving part second jaw auxiliary rotation shaft. Each of the rotation shafts 241, 242, 243, 244, 245, and 246 may be fitted into one or more pulleys, which will be described in detail later.

In addition, the driving part 200 according to an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be provided in the form of a rotatable flat plate, in which one or more coupling holes, to which a motor (not shown) may be coupled, may be formed.

The motor coupling parts 251, 252, 253, and 254 of the driving part 200 described above are coupled to motors (not shown) formed in the robot arm units 21, 22, and 23, respectively, so that the driving part 200 is operated by driving the motors (not shown).

In addition, the driving part 200 according to an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, and the pulley 221 and the pulley 222 may function as driving part second jaw pulleys, and these components may be collectively referred to as driving part jaw pulleys.

Here, it is illustrated in the drawings that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys in the driving part may be associated with a yaw motion, and one group of pulleys in the driving part may be associated with an actuation motion. Thus, the pulley 211 and the pulley 212 may be collectively referred to as driving part driving pulleys. In addition, in the other pulleys, one group of pulleys may also be associated with a yaw motion, and one group of pulleys may also be associated with an actuation motion.

The pulley 213 and the pulley 214 may function as driving part first jaw auxiliary pulleys, and the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, and the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, and the pulley 227 and the pulley 228 may function as driving part second jaw second relay pulleys, and these components may be collectively referred to as driving part second jaw relay pulleys. Meanwhile, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, it is illustrated in the drawings that two pulleys are paired to form the driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with not just two pulleys but also with three or more pulleys.

Meanwhile, the pulley 219 and the pulley 220 may function as driving part first jaw satellite pulleys, and the pulley 229 and the pulley 230 may function as driving part second jaw satellite pulleys, and these two components may be collectively referred to as driving part satellite pulleys.

A plurality of rotation shafts including the driving part first jaw rotation shaft 241, the driving part second jaw rotation shaft 242, the driving part pitch rotation shaft 243, the driving part roll rotation shaft 244, the driving part first jaw auxiliary rotation shaft 245, and the driving part second jaw auxiliary rotation shaft 246 may be formed on a first surface of a base plate 201. In addition, a plurality of relay pulleys 202 are formed on the first surface of the base plate 201, and may serve to redirect the wires 301, 302, 303, 304, 305, and 306 entering the driving part 200 through the connection part 310 toward the pulley 231.

Further, the connection part 310 in the form of a shaft is coupled to a second surface of the base plate 201 opposite to the first surface, and the first jaw motor coupling part 251, the second jaw driving motor coupling part 252, the pitch driving motor coupling part 253, and the roll driving motor coupling part 254, to which the motors (not shown) for driving the pulleys are coupled, may be formed on the second surface.

Here, each rotation shaft and each motor coupling part may be directly connected or indirectly connected to each other via a gear.

In an example, by directly coupling the first jaw motor coupling part 251 to the driving part first jaw rotation shaft 241, when the first jaw motor coupling part 251 coupled to a first jaw driving motor (not shown) is rotated, the driving part first jaw rotation shaft 241 directly coupled to the first jaw motor coupling part 251 may be rotated together. Similarly, by directly coupling the second jaw driving motor coupling part 252 to the driving part second jaw rotation shaft 242, when the second jaw driving motor coupling part 252 coupled to a second jaw driving motor (not shown) is rotated, the driving part second jaw rotation shaft 242 directly coupled to the second jaw driving motor coupling part 252 may be rotated together.

In another example, when viewed from a plane perpendicular to the driving part pitch rotation shaft 243, the pitch driving motor coupling part 253 and the driving part pitch rotation shaft 243 may be disposed to be spaced apart from each other by a certain extent. In addition, the pitch driving motor coupling part 253 and the driving part pitch rotation shaft 243 may be connected to each other by the gears 261 and 263, which are pitch driving gears.

Similarly, when viewed from a plane perpendicular to the driving part roll rotation shaft 244, the roll driving motor coupling part 254 and the driving part roll rotation shaft 244 may be disposed to be spaced apart from each other by a certain extent. In addition, the roll driving motor coupling part 254 and the driving part roll rotation shaft 244 may be connected to each other by the gears 263 and 264, which are roll driving gears.

As such, some motor coupling parts are configured to be directly connected to the rotation shafts, respectively, and the remaining motor coupling parts are configured to be indirectly connected to the rotation shafts, respectively, because the coupling position and direction between the multi-joint type surgical instrument 30 and the slave robot 20 should be considered. That is, the rotation shaft that is not affected by the coupling position with the slave robot 20 is directly connected to the motor coupling part, whereas the rotation shaft that may cause interference with the coupling position with the slave robot 20 may be indirectly connected to the motor coupling part.

It is illustrated in the drawings that the first jaw motor coupling part 251 and the second jaw driving motor coupling part 252 are directly connected to the rotation shafts, respectively, and the pitch driving motor coupling part 253 and the roll driving motor coupling part 254 are indirectly connected, respectively, through the gears, but an embodiment of the present disclosure is not limited thereto, and various configurations are possible according to the coupling position and direction with the slave robot 20.

The pulleys 211 and 212, which are driving part first jaw pulleys, may be coupled to the driving part first jaw rotation shaft 241. Here, the pulleys 211 and 212 may be formed to rotate together with the driving part first jaw rotation shaft 241.

In addition, the driving part first jaw auxiliary rotation shaft 245 may be disposed in a region adjacent to the driving part first jaw rotation shaft 241. The pulleys 213 and 214, which are driving part first jaw auxiliary pulleys, may be coupled to the driving part first jaw auxiliary rotation shaft 245. Here, the pulleys 213 and 214 may be formed to be rotatable around the driving part first jaw auxiliary rotation shaft 245.

Here, it is illustrated in the drawings that the driving part first jaw pulley is formed of two pulleys 211 and 212, the wire 301 is coupled to one pulley 211, and the wire 305 is coupled to the other pulley 212. However, an embodiment of the present disclosure is not limited thereto, and the driving part first jaw pulley may be formed of one pulley, and both the wires 301 and 305 may be coupled to the one pulley.

As described above, the driving part first jaw rotation shaft 241 is coupled to the first jaw driving motor (not shown) by the first jaw motor coupling part 251, and thus, when the first jaw driving motor (not shown) rotates for driving the first jaw 101, the pulleys 211 and 212, which are driving part first jaw pulleys, are rotated together with the driving part first jaw rotation shaft 241, so that the wires 301 and 305, which are first jaw wires, are pulled or released.

The pulleys 221 and 222, which are driving part second jaw rotation shafts, may be coupled to the driving part second jaw rotation shaft 242. Here, the pulley 221 and the pulley 222 may be formed to rotate together with the driving part second jaw rotation shaft 242.

In addition, the driving part second jaw auxiliary rotation shaft 246 may be disposed in a region adjacent to the driving part second jaw rotation shaft 242. The pulleys 223 and 224, which are driving part second jaw auxiliary pulleys, may be coupled to the driving part first jaw auxiliary rotation shaft 245. Here, the pulleys 223 and 224 may be formed to be rotatable around the driving part second jaw auxiliary rotation shaft 246.

Here, it is illustrated in the drawings that the driving part second jaw pulley is formed of two pulleys 221 and 222, the wire 302 is coupled to one pulley 221, and the wire 306 is coupled to the other pulley 222. However, an embodiment of the present disclosure is not limited thereto, and the driving part second jaw pulley may be formed of one pulley, and both the wires 302 and 306 may be coupled to the one pulley.

As described above, the driving part second jaw rotation shaft 242 is coupled to the second jaw driving motor (not shown) by the second jaw driving motor coupling part 252, and thus, when the second jaw driving motor (not shown) rotates for driving the second jaw 102, the pulley 221 and the pulley 222, which are driving part second jaw pulleys, are rotated together with the driving part second jaw rotation shaft 242, so that the wires 302 and 306, which are second jaw wires, are pulled or released.

The pulley 231, which is a driving part pitch pulley, may be coupled to the driving part pitch rotation shaft 243. Here, the pulley 231 may be formed to rotate together with the driving part pitch rotation shaft 243.

As described above, the driving part pitch rotation shaft 243 is coupled to a pitch driving motor (not shown) by the pitch driving motor coupling part 253, and thus, when the pitch driving motor (not shown) rotates for a pitch motion, the wires 303 and 304, which are pitch wires, are pulled or released as the pulley 231, which is a driving part pitch pulley, is rotated together with the driving part pitch rotation shaft 243.

Meanwhile, the pulley 215, the pulley 216, the pulley 217, the pulley 218, the pulley 225, the pulley 226, the pulley 227, and the pulley 228, which are driving part relay pulleys, may be formed to be rotatable around the driving part pitch rotation shaft 243 by inserting the driving part pitch rotation shaft 243 therethrough. Here, the pulley 215, the pulley 216, the pulley 217, and the pulley 218, which are driving part first jaw relay pulleys, may be disposed on one surface side of the pulley 231 that is a pitch pulley, and the pulley 225, the pulley 226, the pulley 227, and the pulley 228, which are driving part second jaw relay pulleys, may be disposed on the other surface side of the pulley 231.

Viewed from another perspective, along the driving part pitch rotation shaft 243, the pulleys 225 and 226, which are driving part second jaw first relay pulleys, the pulleys 227 and 228, which are driving part second jaw second relay pulleys, the pulley 231, which is a driving part pitch pulley, and the pulleys 217 and 218, which are driving part first jaw second relay pulleys, and the pulleys 215 and 216, which are driving part first jaw first relay pulleys, are sequentially stacked and formed.

In addition, the pitch-yaw connector 232 may be coupled to the driving part pitch rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys to allow the driving part satellite pulleys to be revolved around the driving part pitch rotation shaft 243 when the pulley 231 is rotated. This will be described in detail later.

Here, the pitch-yaw connector 232 may be formed to rotate together with the driving part pitch rotation shaft 243. That is, the pulley 231 and the pitch-yaw connector 232 may be coupled to the driving part pitch rotation shaft 243, and may be rotated together with the driving part pitch rotation shaft 243.

Here, the pitch-yaw connector 232 may be described as being formed in an approximately Y-shape as shown in FIG. 12, or the pitch-yaw connector 232 may be described as being formed in a shape in which at least two extension portions 232a and 232b are formed to extend from the center thereof. In addition, a driving part first jaw satellite pulley central shaft 233 and a driving part second jaw satellite pulley central shaft 234 may be formed at end portions of the extension portions 232a and 232b, respectively.

In addition, the pulleys 219 and 220, which are driving part first jaw satellite pulleys, may be coupled to the driving part first jaw satellite pulley central shaft 233, and the pulleys 229 and 230, which are driving part second jaw satellite pulleys, may be coupled to the driving part second jaw satellite pulley central shaft 234.

As a result, when the pulley 231, which is a driving part pitch pulley, is rotated together with the driving part pitch rotation shaft 243, the pulley 219, the pulley 220, the pulley 229, and the pulley 230, which are driving part satellite pulleys, are revolved around the driving part pitch rotation shaft 243. In other words, it may be said that the driving part first jaw satellite pulley central shaft 233 and the driving part second jaw satellite pulley central shaft 234 are rotated around the driving part pitch rotation shaft 243 while maintaining a constant distance from the driving part pitch rotation shaft 243 in a state in which the driving part first jaw satellite pulley central shaft 233 and the driving part second jaw satellite pulley central shaft 234 are spaced apart from the driving part pitch rotation shaft 243 by a certain extent.

That is, the driving part satellite pulley is formed to be movable relative to the driving part relay pulley and the driving part pitch rotation shaft 243 so that a relative position of the driving part satellite pulley with respect to the driving part relay pulley and the driving part pitch rotation shaft 243 may be changed. On the other hand, the relative positions of the driving part pitch pulley 231 and the driving part relay pulley remain constant.

In addition, when the pulley 231, which is a driving part pitch pulley, is rotated around the driving part pitch rotation shaft 243, the pulley 219, the pulley 220, the pulley 229, and the pulley 230, which are driving part satellite pulleys, are moved relative to the pulley 231, which is a driving part pitch pulley, so that the overall lengths of the wire 301, the wire 302, the wire 305, and the wire 306, which are jaw wires, in the driving part 200 are changed.

The wire 301, which is a first jaw wire, is connected to the end tool 100 through the connection part 310 after being sequentially wound to make contact with at least portions of the pulley 211, the pulley 213, the pulley 215, the pulley 219, and the pulley 217 in a state in which one end portion of the wire 301 is coupled to the pulley 211 by the first jaw wire-driving part coupling member (not shown).

Viewed from another perspective, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

Viewed from another perspective, the wire 301, which is a first jaw wire, enters the driving part 200 after passing through the end tool 100 and the connection part 310, and then is fixedly coupled to the pulley 211, which is a driving part first jaw pulley after being sequentially wound around the pulley 217, the pulley 219, the pulley 215, and the pulley 213.

Meanwhile, the wire 305, which is a first jaw wire, is connected to the end tool 100 through the connection part 310 after being sequentially wound to make contact with at least portions of the pulley 212, the pulley 214, the pulley 216, the pulley 220, and the pulley 218 in a state in which one end portion of the wire 305 is coupled to the pulley 212 by the first jaw wire-driving part coupling member (not shown).

The wire 302, which is a second jaw wire, is connected to the end tool 100 through the connection part 310 after being sequentially wound to make contact with at least portions of the pulley 221, the pulley 223, the pulley 225, the pulley 229, and the pulley 227 in a state in which one end portion thereof is coupled to the pulley 221 by the second jaw wire-driving part coupling member (not shown).

Meanwhile, the wire 306, which is a second jaw wire, is connected to the end tool 100 through the connection part 310 after being sequentially wound to make contact with at least portions of the pulley 222, the pulley 224, the pulley 226, the pulley 230, and the pulley 228 in a state in which one end portion thereof is coupled to the pulley 222 by the second jaw wire-driving part coupling member (not shown).

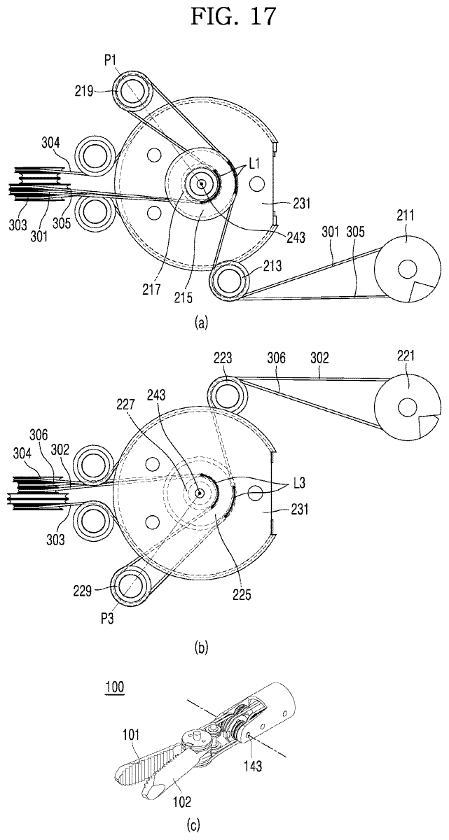

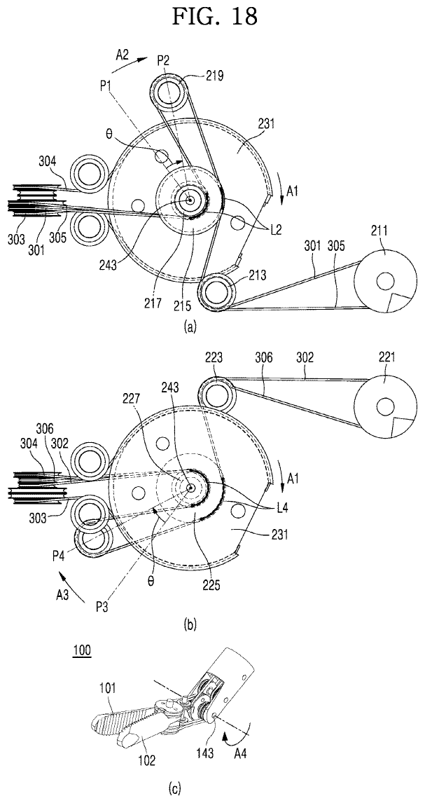

FIGS. 17 and 18 are views illustrating a pitch motion of the multi-joint type surgical instrument illustrated in FIG. 6. Here, for convenience of description, only the pulleys and wires related to the rotation of the first jaw are illustrated in (a) of FIGS. 17 and 18, and only the pulleys and wires related to the rotation of the second jaw are illustrated in (b) of FIGS. 17 and 18. In addition, (c) of FIGS. 17 and 18 illustrate a pitch motion of the end tool according to a pitch motion of the driving part.

Here, in the multi-joint type surgical instrument 30 according to an embodiment of the present disclosure, when the driving part satellite pulley is moved relative to the driving part relay pulley, which causes the overall length of the jaw wire to be changed in the driving part 200, allowing the end tool 100 to perform a pitch motion. In particular, in the multi-joint type surgical instrument 30 according to an embodiment of the present disclosure, when the driving part pitch pulley 231 is rotated, which causes the driving part satellite pulley to be revolved around the (common) rotation shaft of the driving part relay pulley and the driving part pitch pulley 231 so that a path length of the jaw wire wound around the driving part relay pulley is changed, allowing the end tool to perform a pitch motion.

Specifically, when a motion compensation for the pitch motion is not separately performed in the driving part, the pitch motion itself cannot be performed in the end tool.

Meanwhile, in order for the end tool to perform a pitch motion, the wires 301 and 305 should be further wound around the pulley 113 by ΔSpitch and the wires 302 and 306 should be further unwound from the pulley 114 by ΔSpitch. However, when such compensation is not performed in the driving part, the pitch motion itself cannot be performed in the end tool.

In order to perform motion compensation for the pitch motion as described above, in the multi-joint type surgical instrument 30 according to an embodiment of the present disclosure, the driving part pitch pulleys are rotated while the driving part satellite pulleys are revolved, so that the jaw wires are wound around or released from the driving part relay pulley, which allows the movement of the jaw wires to be compensated for by the rotation of the driving part pitch pulley 231.

In other words, when the pulley 231, which is a driving part pitch pulley, is rotated together with the driving part pitch rotation shaft 243, the driving part satellite pulleys are revolved around the driving part pitch rotation shaft 243. In addition, as the driving part satellite pulleys are revolved around the driving part pitch rotation shaft 243, the jaw wire wound around the driving part relay pulley is changed in length. That is, the jaw wire wound at the end tool 100 side due to the rotation of the pulley 231 is released by the same amount at the driving part 200 side, and the jaw wire unwound at the end tool 100 side is wound by the same amount at the driving part 200 side, so that the pitch motion does not affect the yaw motion.

Viewed from another perspective, when the end tool performs a pitch motion due to the rotation of the driving part pitch pulley 231, the jaw wire (responsible for the yaw and actuation motions) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may be described that in the present disclosure, in order to compensate for the movement of the jaw wire, when the end tool performs the pitch motion, the overall length of the jaw wire in the driving part is changed while the driving part satellite pulley is moved relative to the driving part relay pulley, so that the jaw wire is released (or pulled) at the end tool side as much as the jaw wire is pulled (or released) at the driving part side, thereby compensating for the movement of the jaw wire when the end tool performs the pitch motion.

Hereinafter, the pitch motion will be described in more detail.

When the pulley 231, which is a driving part pitch pulley, is rotated in the direction of an arrow A1 (i.e., in the clockwise direction in the drawing) in order for the pitch motion, the pitch-yaw connector 232 (see FIG. 10) is rotated in the direction of the arrow A1 together with the pulley 231, and thus, the pulleys 219 and 220, which are driving part satellite pulleys fixedly coupled to the pitch-yaw connector 232 (see FIG. 10), are revolved as a whole in the direction of an arrow A2 of FIG. 18A (i.e., in the clockwise direction in the drawing) around the driving part pitch rotation shaft 243 by θ. That is, when the pulley 231 is rotated, the pulleys 219 and 220 are revolved by θ from the position of P1 of (a) of FIG. 17 to the position of P2 of (a) of FIG. 18. Viewed from another perspective, it may be described that when the driving part pitch pulley 231 is rotated, the driving part satellite pulley is moved in conjunction with the driving part pitch pulley 231.

At the same time, when the pulley 231, which is a driving part pitch pulley, is rotated in the direction of the arrow A1 (i.e., in the clockwise direction in the drawing), the pitch-yaw connector 232 (see FIG. 10) is rotated in the direction of the arrow A1 together with the pulley 231, and thus, the pulleys 229 and 230, which are driving part satellite pulleys fixedly coupled to the pitch-yaw connector 232 (see FIG. 10), are revolved as a whole in the direction of an arrow A3 of FIG. 18B (i.e., in the clockwise direction in the drawing) around the driving part pitch rotation shaft 243 by θ. That is, when the pulley 231 is rotated, the pulleys 229 and 230 are revolved by θ from the position of P3 of (b) of FIG. 17 to the position of P4 of (b) of FIG. 18. Viewed from another perspective, it may be described that when the driving part pitch pulley 231 is rotated, the driving part satellite pulley is moved in conjunction with the driving part pitch pulley 231.

Meanwhile, in this case, the positions of the pulley 215, the pulley 216, the pulley 217, the pulley 218, the pulley 225, the pulley 226, the pulley 227, and the pulley 228, which are driving part relay pulleys coupled to the driving part pitch rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley is revolved, and thus, the length of each wire wound around the driving part relay pulley, that is, the path length, is changed. Here, since the driving part relay pulley includes the pulley 215, which is a driving part first jaw first relay pulley, and the pulley 217, which is a driving part first jaw second relay pulley, the path length also means the sum of the length of the wire 301 wound around the pulley 215 and the length of the wire 301 wound around the pulley 217 (or, the sum of the length by which the wire 305 is wound around the pulley 216 and the length by which the wire 305 is wound on the pulley 218).

That is, as compared to a path length L1 by which the wires 301 and 305, which are first jaw wires, wound around the driving part relay pulleys at the position of (a) of FIG. 17, a path length L2 by which the first jaw wires wound around the driving part relay pulleys at the position of FIG. 18A is reduced, and thus, the first jaw wires are further released at the driving part 200 side by the reduced path length (L1-L2). That is, the overall lengths of the wires 301 and 305, which are first jaw wires, in the driving part 200 are reduced. In addition, as the overall length of the first jaw wire in the driving part 200 is reduced, the overall length of the first jaw wire in the end tool 100 is increased as much as the first jaw wire is unwound.

In contrast, when the pulley 231, which is a driving part pitch pulley, is rotated in the direction of the arrow A1, as compared to a path length L3 by which the wires 302 and 306, which are second jaw wires, wound around the driving part relay pulleys at the position of (b) of FIG. 17, a path length L4 by which the second jaw wires wound around the driving part relay pulleys at the position of (b) of FIG. 18 is increased, and the second jaw wires are further pulled at the driving part 200 side by as much as the increased path length (L4-L3). That is, the overall lengths of the wires 302 and 306, which are second jaw wires, in the driving part 200 are increased. In addition, as the overall length of the second jaw wire in the driving part 200 is increased, the overall length of the second jaw wire in the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley 231 and the driving part relay pulley. In addition, due to the relative movement of the driving part satellite pulley, the overall length of the first jaw wire in the driving part 200 is reduced, and the overall length of the first jaw wire in the end tool 100 is increased. At the same time, due to the relative movement of the driving part satellite pulley, the overall length of the second jaw wire in the driving part 200 is increased, and the overall length of the second jaw wire in the end tool 100 is reduced.

As a result, when the pulley 231, which is a driving part pitch pulley, is rotated in the direction of the arrow A1, the wires 301 and 305, which are two strands of the first jaw wire, are released and the wires 302 and 306, which are two strands of the second jaw wire, are pulled when viewed from the end tool 100 side, so that the end tool 100 performs a pitch motion in the direction of an arrow A4 around the rotation shaft 143.

Here, the term “path length” may be defined as a length of the jaw wire from a point at which the jaw wire enters the driving part first relay pulley to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

Viewed from another perspective, the path length may be defined as the length of the jaw wire from an initial contact point of the jaw wire with the driving part relay pulley to a final contact point of the jaw wire with the driving part relay pulley on a deployment path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed while the driving part satellite pulley is moved relative to the driving part relay pulley, the overall length of the jaw wire in the driving part 200 is also changed. In addition, as the overall length of the jaw wire in the driving part 200 is changed, the overall length of the jaw wire in the end tool 100 is also changed. However, it may be said that since the overall length of the jaw wire in the end tool 100 is also increased (or reduced) by as much as the overall length of the jaw wire increased (reduced) in the driving part 200, a total length of the jaw wire is not changed (assuming that elastic deformation or the like is not considered).

As a result, when the driving part pitch pulley 231 is rotated, the wire 301/wire 305, which are first jaw wires, are released at the driving part 200 side by as much as the wire 301/wire 305, which are first jaw wires, are pulled at the end tool 100 side, as a result, a pitch motion is enabled.

Meanwhile, as described above, the end tool 100 of the multi-joint type surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 which are pitch wires.

Accordingly, when the pulley 231, which is a driving part pitch pulley, is rotated in the direction of the arrow A1, due to the rotation of the pulley 231, the wire 304 is wound around the pulley 231 and the wire 303 is released from the pulley 231. Accordingly, the pulley 131, which is an end tool pitch pulley connected to the other sides of the wires 303 and 304, is rotated in the direction of the arrow A2 around the rotation shaft 143, so that the pitch motion may be more surely and reliably performed.

Here, among the pulleys that are rotated around the rotation shaft 143, which is an end tool pitch rotation shaft, the pulley 131, which is an end tool pitch pulley in contact with the wires 303 and 304 that are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306 that are jaw wires.

In this case, when the rotation shaft 143 is rotated, the lengths of the wires wound around or unwound from the respective pulleys are different from each other. For example, when a diameter of the end tool pitch pulley is 6 φ, a diameter of the end tool jaw pitch main pulley is 4 φ, and the rotation shaft 143 is rotated by 90°, a length of the pitch wire wound around the end tool pitch pulley is 1.5 π, whereas a length of the jaw wire wound around the end tool jaw pitch main pulley may be 1 π.

From this perspective, the length of the wire wound around or unwound from the pulley may be defined as “rotation amount”. The rotation amount is a concept different from a rotation angle, and may be calculated as (diameter*rotation angle/360°*π).

In this case, since essentially the pulley 231, which is a driving part pitch pulley, is directly connected to the pulley 131, which is an end tool pitch pulley, by the wires 303 and 304, which are pitch wires, the rotation amount of the driving part pitch pulley 231 is the same as that of the end tool pitch pulley. That is, the pitch wire is released from or wound around the end tool pitch pulley by as much as the pitch wire is wound around or released from the driving part pitch pulley 231.

Meanwhile, a relation of (diameter of end tool pitch pulley:diameter of end tool jaw pitch main pulley)=(rotation amount of wire wound around end tool pitch pulley:rotation amount of wire wound around end tool jaw pitch main pulley) may be established.

As described above, when, in the end tool 100, the length of the pitch wire wound around the end tool pitch pulley is different from the length of the jaw wire wound around the end tool jaw pitch main pulley, in the driving part 200, the length of the pitch wire to be released should be different from the length of the jaw wire to be released by the same proportion.

To this end, the relationship of (diameter of end tool pitch pulley:diameter of end tool jaw pitch main pulley)=(diameter of driving part pitch pulley:diameter of driving part relay pulley) may be established.

For example, when a ratio of (diameter of end tool pitch pulley:diameter of end tool jaw pitch main pulley) is 6:4, a ratio of (diameter of driving part pitch pulley:diameter of driving part relay pulley) may also be 6:4. According to this ratio, the diameter of the driving part pitch pulley may be 9 φ, and the diameter of the driving part relay pulley may be 6 φ.

However, here, the driving part relay pulley may include two or more pulleys including the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 6φ, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ), among others. Here, it is illustrated in the drawings that the diameter of the pulley 215, which is a driving part first relay pulley, is 4 φ, and the diameter of the pulley 217, which is the driving part second relay pulley, is 2 φ.

In addition, it may be described that rotation amount of driving part first relay pulley plus the rotation amount of driving part second relay pulley is proportional to the rotation amount of the driving part pitch pulley.

However, although the ratio of (diameter of end tool pitch pulley:diameter of end tool jaw pitch main pulley) may not exactly match the ratio of (diameter of driving part pitch pulley:diameter of driving part relay pulley), when the pulley diameters are selected to make these ratios similar, the object of the present disclosure, which is to compensate for the movement of the jaw wire with the rotation of the driving part pitch pulley, can be achieved to some extent.

The process of the final pitch motion will be described again as follows.

Hereinafter, a case in which the diameter of the end tool pitch pulley is 6 φ, the diameter of the end tool jaw pitch main pulley is 4 φ, the diameter of the driving part pitch pulley is 9 φ, and the diameter of the driving part relay pulley is 6 φ will be described as an example.

First, for a pitch motion, the pulley 231, which is a driving part pitch pulley of the driving part 200, is rotated by 60° to wind the wire 304, which is a pitch wire, while releasing the wire 303. At this time, the length of the wire 303/wire 304 wound and unwound is 1.5 π.

Accordingly, as the wire 304 is pulled by 1.5 π and the wire 303 is released by 1.5 π in the end tool 100, the pulley 131, which is an end tool pitch pulley, is rotated by 90° corresponding to 1.5 π.

Meanwhile, when the pulley 131 is pitch-rotated around the rotation shaft 143, the jaws 101 and 102 and the pulley 111/pulley 112 are also pitch-rotated around the rotation shaft 143. Accordingly, the wires 301 and 305, which are first jaw wires coupled to the pulley 111, are both pulled, and the wires 302 and 306, which are second jaw wires coupled to the pulley 121, are both released. At this time, the angles by which the end tool pitch pulley and the end tool jaw pitch main pulley are rotated are equal to each other and measure 90°, and thus, the length of the jaw wires wound around or released from the end tool jaw pitch main pulley becomes 1 π.

Meanwhile, since the pulley 231 and the pulley 219/pulley 220 are rigidly connected by the pitch-yaw connector 232, when the pulley 231 is rotated by 60° around the driving part pitch rotation shaft 243, the pulley 219/pulley 220 are revolved by 600 around the driving part pitch rotation shaft 243.

In addition, as described above, as the pulley 219/pulley 220 are revolved, the jaw wires are wound around or released from the pulley 215 and the pulley 216, whose combined diameter is 6 φ, by 1 π corresponding to a revolution angle of 60°. That is, the wires 301 and 305, which are first jaw wires, are released as a whole, and the wires 302 and 306, which are second jaw wires, are pulled as a whole.

In other words, the overall path lengths of the wires 301 and 305 wound around the pulley 215, the pulley 216, the pulley 217, and the pulley 218, which are driving part first jaw relay pulleys, are reduced, and the wires 301 and 305 are released by as much as the reduced path length. In addition, the overall path lengths of the wires 302 and 306 wound around the pulley 225, the pulley 226, the pulley 227, and the pulley 228, which are driving part second jaw relay pulleys, are increased, and the wires 302 and 306 are pulled by as much as the increased path length.

That is, the wires 301 and 305, which are first jaw wires, are released at the driving part 200 side by as much as the wires 301 and 305 are pulled at the end tool 100 side, thereby compensating for the movement of the jaw wire due to the pitch motion. Similarly, the wires 302 and 306, which are second jaw wires, are released at the driving part 200 side by as much as the wires 302 and 306 are pulled at the end tool 100 side, thereby compensating for the movement of the jaw wire due to the pitch motion.

As a result, by releasing (or pulling) the jaw wires at the driving part 200 side by as much as a length equal to the length by which the jaw wires are wound around (or released from) the end tool 100 side in response to the pitch motion, the pitch motion can be performed independently without affecting the rotation of the jaw around the yaw shaft.

That is, when the driving part pitch pulley 231 and the driving part satellite pulley are rigidly connected, and the driving part pitch pulley 231 is rotated around the driving part pitch rotation shaft 243, the path length of the jaw wire wound around the driving part relay pulley is changed as the driving part satellite pulley is revolved around the driving part pitch rotation shaft 243. In addition, the change in the path length of the jaw wire compensates for the movement of the jaw wires at the end tool side due to the pitch motion, as a result, the pitch motion is independently performed.

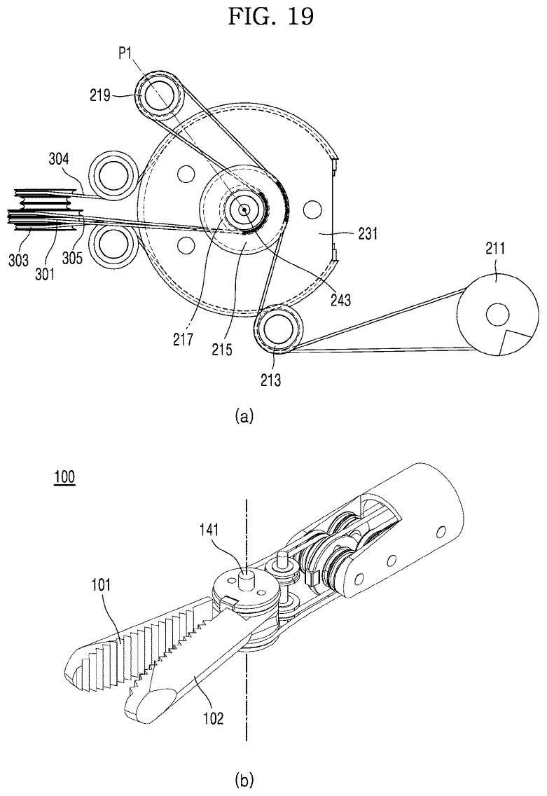

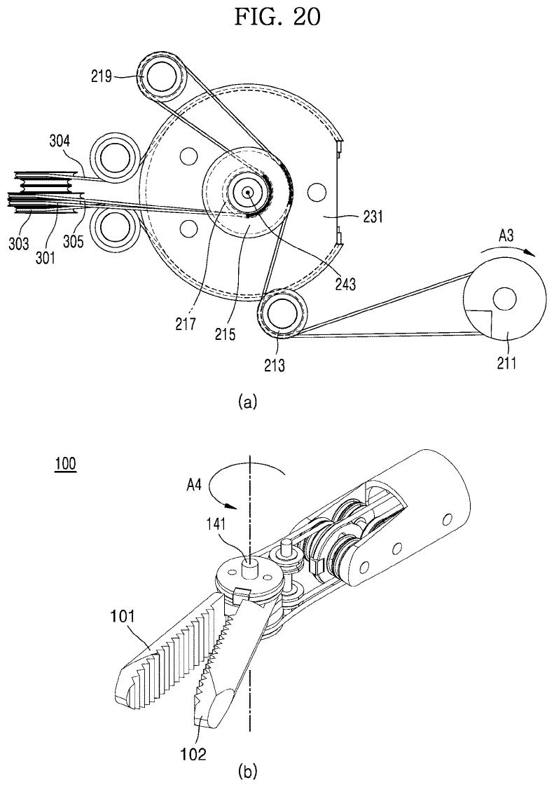

FIGS. 19 and 20 are views illustrating a yaw motion of the multi-joint type surgical instrument illustrated in FIG. 6.

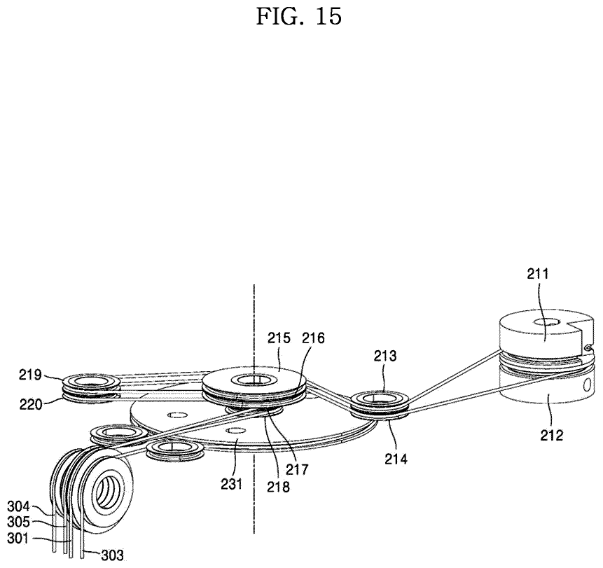

Referring to FIGS. 15, 16, 19, and 20 and the like, when the pulley 211, which is a driving part first jaw pulley, is rotated in the direction of an arrow A3 for a yaw motion, one of the wires 301 and 305, which are first jaw wires, is wound around the pulley 211 and the other one thereof is released from the pulley 211 in response to the rotation of the pulley 211. Accordingly, the pulley 111, which is an end tool first jaw pulley connected to the opposite side of the wires 301 and 305, is rotated in the direction of as arrow A4, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 301 and 305 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

Similarly, when the pulley 221, which is a driving part second jaw pulley, is rotated for a yaw motion, in response to the rotation of the pulley 221, one of the wires 302 and 306, which are second jaw wires, is wound around the pulley 221 and the other one thereof is released from the pulley 221. Accordingly, the pulley 121, which is an end tool second jaw pulley connected to the opposite side of the wires 302 and 306, is rotated in one direction, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 302 and 306 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

As a result, the overall lengths of the wire 301, the wire 302, the wire 305, and the wire 306, which are jaw wires, in the driving part 200 remain constant even when the pulley 211 or pulley 221, which is a driving part jaw pulley, is rotated for the yaw or actuation motion.

As described above, in the multi-joint type surgical instrument 30 according to an embodiment of the present disclosure, when the driving part pitch pulley 231 is rotated, the driving part satellite pulley is revolved around the rotation shaft of the driving part pitch 231 pulley to change the path length of the jaw wire wound around the driving part relay pulley, and the jaw wire is wound or released in response to the rotation of the driving part pitch pulley 231, so that the movement of the jaw wire due to the pitch drive may be offset or compensated, and as a result, the effect of separating the pitch motion and the yaw motion can be obtained.

However, the pitch motion and the yaw motion are not limited to being mechanically separated from each other as described above, and can be separated and performed independently by the processor according to an embodiment of the present disclosure.

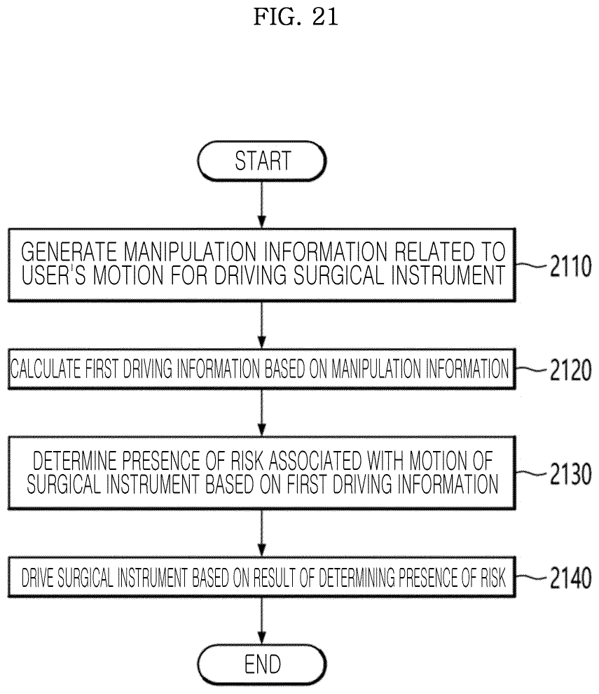

FIG. 21 is a flowchart for describing an example of a method of driving a surgical instrument according to an embodiment.

Referring to FIG. 21, the method of driving a surgical instrument includes operations that are processed by the user terminal 2000 or the processor 2011 illustrated in FIGS. 1 and 2A. Thus, the contents described above regarding the user terminal 2000 or the processor 2011 illustrated in FIGS. 1 and 2A may also be applied to the method of driving the surgical instrument of FIG. 21 even when omitted below.

In addition, as described above with reference to FIGS. 1 and 2B, at least one of the operations of the method of driving a surgical instrument of FIG. 21 may be processed by the server 3000 or the processor 3011.

In addition, as described above with reference to FIGS. 3 to 5, at least one of the operations of the method of driving a surgical instrument of FIG. 21 may be processed by the master robot 10, the slave robot 20, or the multi-joint type surgical instrument 30.

In operation 2110, the processor 2011 generates manipulation information related to a user's motion for driving the surgical instrument. Specifically, the processor 2011 generates manipulation information related to a member that allows a user to manipulate the position and function of the surgical instrument.

The manipulation information may be information generated by using position information of the member that allows a user to manipulate the position and function of the surgical instrument. For example, the manipulation information may include a difference between position and orientation information of the member, which allows a user to manipulate the position and function of the surgical instrument in a coordinate system before a user's motion, and position and orientation information of the member, which allows a user to manipulate the position and function of the surgical instrument in a coordinate system after the user's motion.

Meanwhile, the processor 2011 may generate the manipulation information based on the position and orientation information of the member that allows a user to manipulate the position and function of the surgical instrument.

In an example, an inverse matrix and a multiplication operation through Equation 1 below may be used to calculate the difference in manipulation information. Here, the position and orientation information in the coordinate system before the user's motion may be position and orientation information in a coordinate system initialized before the user's motion.

$${T}_{d e v i c e} = {T}_{0}^{- 1} {T}_{curr} \qquad [ Equation 1 ]$$

According to Equation 1, the processor 2011 may generate manipulation information T_{device} for driving the surgical instrument based on position and orientation information T₀ of a member, which allows a user to manipulate the position and function of the surgical instrument, in an initialized coordinate system and position and orientation information T_{curr} of the member, which allows a user to manipulate the position and function of the surgical instrument, in a coordinate system after a user's motion.

However, examples of the method of generating the manipulation information are not limited to the above description.

In operation 2120, the processor 2011 calculates first driving information based on the manipulation information. For example, the processor 2011 may calculate first driving information related to a motion of the surgical instrument based on the manipulation information related to the user's motion. Here, the manipulation information includes position and orientation information related to the user's motion, and the first driving information may include position and orientation information related to the motion of the surgical instrument corresponding to the user's motion.

According to an embodiment of the present disclosure, by calculating the first driving information related to the motion of the surgical instrument based on manipulation information related to a user's intuitive motion, the surgical robot can more accurately reflect an intuitive manipulation of the user and perform surgery.

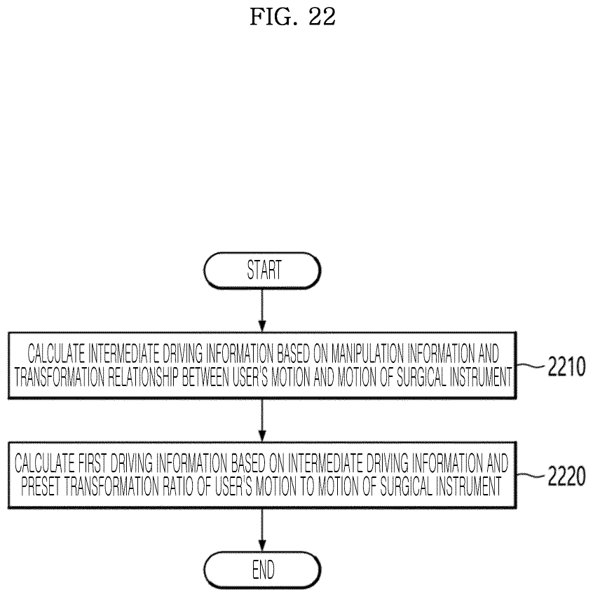

Hereinafter, an example of a method by which the processor calculates the first driving information will be described with reference to FIG. 22.

FIG. 22 is a flowchart for describing an example of the method by which the processor according to an embodiment calculates the first driving information. Referring to FIG. 22, the processor 2011 may calculate intermediate driving information based on the manipulation information and a transformation relationship between the user's motion and the motion of the surgical instrument, and calculate the first driving information based on the intermediate driving information and a preset transformation ratio of the user's motion to the motion of the surgical instrument.

In operation 2210, the processor 2011 calculates the intermediate driving information based on the manipulation information and the transformation relationship between the user's motion and the motion of the surgical instrument. Specifically, the processor 2011 calculates intermediate position information of the surgical instrument based on a transformation relationship between the member, which allows a user to manipulate the position and function of the surgical instrument, and a camera attached to the surgical instrument, calculates intermediate orientation information of the surgical instrument based on a transformation relationship between the member and the surgical instrument, and generates intermediate driving information by using the intermediate position information of the surgical instrument and the intermediate orientation information of the surgical instrument.

For example, the processor 2011 may generate the intermediate driving information by transforming the manipulation information according to Equation 2 below based on rotation matrices R_{device→robot_global}, R_{device→robot_tool}, R_{robot_global→camera_view}.

The rotation matrices refer to matrices that perform a rotational transformation on an arbitrary matrix around an origin or a reference point. For example, the rotation matrices may include matrices R_{device→robot_global}, R_{device→robot_tool}, and R_{robot_global→camera_view} that transform the position and orientation information, which is information in a physical coordinate system of the member that allows a user to manipulate the position and function of the surgical instrument and included in the manipulation information, from the coordinate system of the member that allows a user to manipulate the position and function of the surgical instrument into a coordinate system of the surgical instrument to be driven.

$${T}_{robot} = {f}_{1} ( {T}_{d e v ice} , {R}_{device \to robot\_global} , {R}_{device \to robot\_tool} , {R}_{robot\_global \to camera\_view} ) \qquad [ Equation 2 ]$$

In other words, according to Equation 2, the processor 2011 may generate intermediate driving information T_{robot} related to a motion of the multi-joint type surgical instrument based on a function ƒ₁, the manipulation information T_{device}, a rotation matrix R_{device→robot_global} that transforms from a coordinate system of the user input part into a coordinate system of the multi-joint type surgical instrument, a rotation matrix R_{device→robot_tool} that transforms from the coordinate system of the user input part into a coordinate system of the end tool, and a rotation matrix R_{robot_global→camera_view} that transforms from the coordinate system of the multi-joint type surgical instrument into a coordinate system of the multi-joint type surgical instrument according to the camera attached to the multi-joint type surgical instrument.

Specifically, through the function ƒ₁, the processor 2011 transforms information of a translation vector of the manipulation information T_{device} including a transform matrix into position information in the form of a three-dimensional vector, and then, multiplies the position information by the rotation matrix R_{device→robot_global} (which transforms from the coordinate system of the user input part to the coordinate system of the multi-joint type surgical instrument) and the rotation matrix R_{robot_global→camera_view} (which transforms from the coordinate system of the multi-joint type surgical instrument to the coordinate system of the multi-joint type surgical instrument according to the camera attached to the multi-joint type surgical instrument), to transform the position information into the coordinate system of the multi-joint type surgical instrument according to the camera attached to the multi-joint type surgical instrument, thereby calculating the intermediate position information of the surgical instrument. Here, the same result may be obtained by multiplying the position information by the rotation matrix R_{device→camera_view}, which transforms from the coordinate system of the user input part to the coordinate system of the multi-joint type surgical instrument according to the camera attached to the multi-joint type surgical instrument.

In addition, the processor 2011 transforms the information of the rotation matrix of the manipulation information T_{device} into orientation information in the form of a quaternion, and extracts only orientation values corresponding to x, y, and z axes from four-dimensional element values, and transforms the orientation values into the coordinate system of the end tool by multiplying the orientation information by the rotation matrix R_{device→robot_tool}, which transforms from the coordinate system of the user input part into the coordinate system of the end tool, thereby calculating the intermediate orientation information of the surgical instrument.

The processor 2011 may generate the intermediate driving information T_{robot} in the form of a homogeneous transform matrix by transforming the transformed intermediate position information and intermediate orientation information in the coordinate system of the end tool into rotation matrix information and translation vector information.

However, examples of the method of generating the intermediate driving information are not limited to the above description.

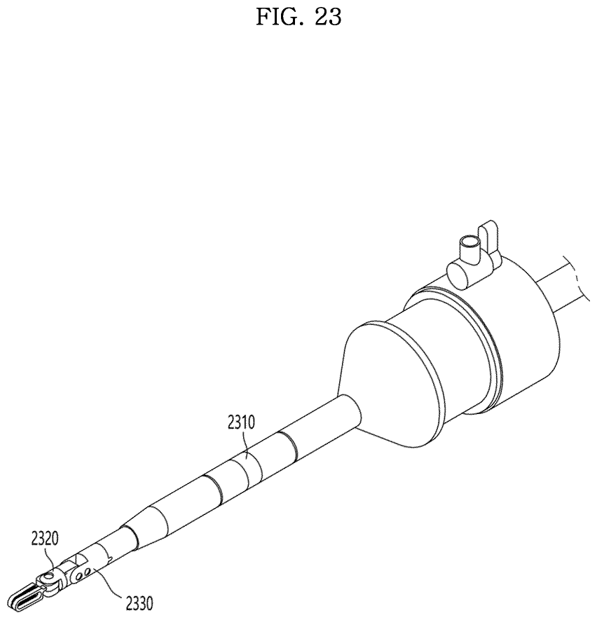

Hereinafter, an example of the transformation relationship between the user's motion and the motion of the surgical instrument will be described with reference to FIG. 23.

FIG. 23 is a view for describing an example of the transformation relationship between the user's motion and the motion of the surgical instrument according to an embodiment, and is a view illustrating an example of the end tool according to an embodiment of the present disclosure.

Referring to FIG. 23, a coordinate system related to the motion of the surgical instrument may have an origin in at least one of a remote center of motion (RCM) 2310 and joints 2320 and 2330 of the surgical instrument.

For example, the coordinate system of the multi-joint type surgical instrument may have the origin at the RCM 2310, and the coordinate system of the end tool may have the origin in at least one of the joints 2320 and 2330 of the surgical instrument. Here, each of the joints 2320 and 2330 of the surgical instrument includes a point that is a center of a rotational motion of the multi-joint type surgical instrument.

According to an embodiment of the present disclosure, based on the above-described origins, the rotation matrix may be used to transform from the coordinate system of the member, which allows a user to manipulate the position and function of the surgical instrument, to the coordinate system of the surgical instrument to be driven.

However, the example of the origin used in the transformation relationship between the user's motion and the motion of a surgical instrument is not limited to the origins of the RCM 2310 and the joints 2320 and 2330 of the surgical instrument illustrated in FIG. 23.

Referring to FIG. 22 again, in operation 2220, the processor 2011 calculates the first driving information based on the intermediate driving information and the preset transformation ratio of the user's motion to the motion of the surgical instrument. Specifically, the processor 2011 may calculate position information of the surgical instrument based on the intermediate position information of the surgical instrument included in the intermediate driving information and a preset position transformation ratio, calculate orientation information of the surgical instrument based on the intermediate orientation information of the surgical instrument included in the intermediate driving information and a preset orientation transformation ratio, and generate the first driving information by using the position information of the surgical instrument and the orientation information of the surgical instrument.

The transformation ratio refers to a ratio related to a degree of motion between the user's motion and the motion of the surgical instrument. Specifically, the transformation ratio may include a ratio regarding a degree of range of motion between a motion of the member that allows a user to manipulate the position and function of the surgical instrument and a corresponding motion of the surgical instrument. For example, the transformation ratio may include a ratio of manipulation of the user input part in response to the user's motion to the motion of the multi-joint type surgical instrument.

Meanwhile, the transformation ratio may be set by a user input, or a transformation ratio previously stored by a user's selection may be set as the transformation ratio. However, the method of setting the transformation ratio is not limited to the above-described example.

According to an embodiment of the present disclosure, by calculating the first driving information based on the intermediate driving information and the preset transformation ratio of the user's motion to the motion of the surgical instrument, the surgical instrument can be manipulated more precisely in response to the user's motion.

For example, the processor 2011 may calculate the first driving information according to Equation 3 below based on the intermediate driving information and the preset transformation ratio of the user's motion to the motion of the surgical instrument.

$${T}_{robot\_target} = {f}_{2} ( {T}_{r o bot} , α , β ) \qquad [ Equation 3 ]$$

According to Equation 3, the processor 2011 may obtain first driving information T_{robot_target} based on a function ƒ₂, intermediate driving information T_{robot}, a position transformation ratio α, and an orientation transformation ratio β. Specifically, the processor 2011 calculates the position information of the surgical instrument by transforming translation vector information of the intermediate driving information T_{robot} into position information in the form of a three-dimensional vector, and then multiplying the position information by the position transformation ratio α. In addition, the processor 2011 calculates the orientation information of the surgical instrument by transforming rotation matrix information of the intermediate driving information T_{robot} into orientation information in the form of a quaternion, extracting only orientation values corresponding to x, y, and z axes from four-dimensional element values, and multiplying the orientation information by the orientation transformation ratio β. In addition, the processor 2011 may obtain the first driving information T_{robot_target} in the form of a homogeneous transform matrix by transforming the position information obtained by multiplying the position transformation ratio α and the orientation information obtained by multiplying the orientation transformation ratio β into the rotation matrix information and the translation vector information.

Meanwhile, the processor 2011 may calculate the orientation information of the surgical instrument according to Equation 4 below.

$$w = s q r t ( 1 - {x}^{2} - {y}^{2} - {z}^{2} ) \qquad [ Equation 4 ]$$

According to Equation 4, the processor 2011 may calculate the orientation information of the surgical instrument by extracting orientation information corresponding to the x-, y-, and z-axes based on the orientation information of the intermediate driving information T_{robot}, multiplying the extracted orientation information by the orientation transformation ratio, and calculating w values based on orientation information x, y, and z corresponding to the x-, y-, and z-axes multiplied by the orientation transformation ratio. Here, the calculated orientation information of the surgical instrument may be orientation information in the form of a quaternion.

However, the example of the method of calculating the first driving information based on the intermediate driving information and the preset transformation ratio of the user's motion to the motion of the surgical instrument is not limited to the above description.

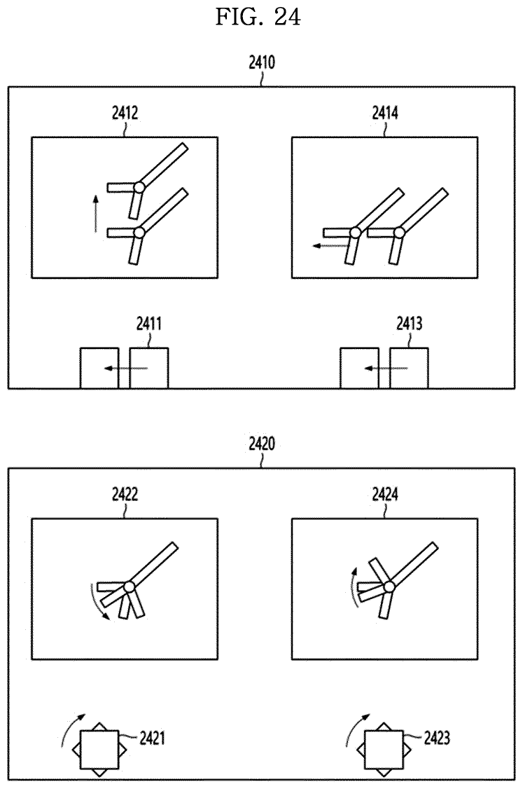

Hereinafter, an example of the surgical instrument operating based on the first driving information calculated according to the method described above will be described with reference to FIG. 24.

FIG. 24 is a view for describing an example of the surgical instrument operating based on the first driving information according to an embodiment.

First, in relation to a manipulation 2410 for moving the surgical instrument, when the surgical instrument is shown on the display member as vertically moving (2412) when the member, which allows a user to manipulate the position and function of the surgical instrument, horizontally moves (2411), it becomes difficult for a user to intuitively manipulate the surgical instrument.

Meanwhile, in relation to the manipulation 2410 of moving the surgical instrument, by driving the surgical instrument based on the first driving information described above, the surgical instrument is shown on the display member as horizontally moving (2414) when the member is horizontally moved (2413). Accordingly, the user can more accurately reflect his or her intuitive manipulation and perform surgery through the surgical robot.

Second, in relation to a manipulation 2420 for moving the surgical instrument, when the surgical instrument is shown on the display member as counterclockwise rotation moving (2422) when the member, which allows a user to manipulate the position and function of the surgical instrument, has clockwise rotation moving (2421), it becomes difficult for a user to intuitively manipulate the surgical instrument.

Meanwhile, in relation to the manipulation 2420 of moving the surgical instrument, by driving the surgical instrument based on the first driving information described above, the surgical instrument is shown on the display member as clockwise rotation moving (2424) when the member has clockwise rotation moving (2423). Accordingly, the user can more accurately reflect his or her intuitive manipulation and perform surgery through the surgical robot.

Referring to FIG. 21 again, in operation 2130, the processor 2011 determines the presence of a risk associated with a motion of the surgical instrument based on the first driving information. For example, the processor 2011 may use the first driving information to determine the presence of a risk in which the surgical instrument cannot operate due to mechanical constraints, or a risk that may cause damage to the surgical instrument or to the patient's body.

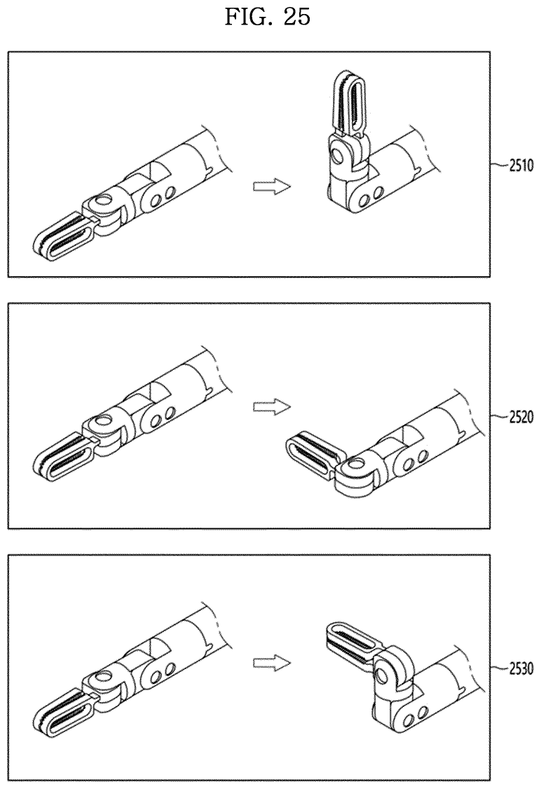

Hereinafter, an example of the motion of a surgical instrument related to the presence of a risk will be described with reference to FIG. 25.

FIG. 25 is a view for describing an example of the range of motion of the surgical instrument according to an embodiment.

Referring to FIG. 25, the processor 2011 may implement various motions by driving the surgical instrument using the first driving information. In an example, the processor 2011 may implement a pitch motion 2510 by driving the end tool according to the method described above using the first driving information. In another example, the processor 2011 may implement a yaw motion 2520 by driving the end tool according to the method described above using the first driving information. In another example, the processor 2011 may implement a motion 2530 including pitch and yaw motions by driving the end tool according to the method described above using the first driving information. Meanwhile, although not shown in the drawings, the processor 2011 may implement a roll motion (not shown) by driving the surgical instrument according to the method described above by using the first driving information.

Here, the range of motion may include at least one of an angle of the pitch motion 2510 of the end tool, an angle of the yaw motion 2520 of the end tool, an angle of the roll motion (not shown) of the surgical instrument, an angle of the motion 2530 including pitch and yaw motions of the end tool.

In a case in which the processor 2011 implements the above-described motions based on the first driving information, a risk in which the surgical instrument does not operate in response to the user's motion due to mechanical constraints, or a risk that may cause damage to the surgical instrument or to the patient's body when the surgical instrument operates in response to the user's motion may occur.

Accordingly, in order to prevent in advance a situation in which the above-described risk occurs, in implementing the various motions 2510, 2520, and 2530 by driving the surgical instrument, the processor 2011 may determine the presence of the risk in which the surgical instrument does not operate in response to the user's motion due to mechanical constraints, or the risk that may cause damage to the surgical instrument or to the patient's body when the surgical instrument operates in response to the user's motion.

Meanwhile, the processor 2011 may determine the presence of a risk in consideration of at least one of a range of motion of the surgical instrument and a singularity of the surgical instrument. For example, the processor 2011 may determine the presence of a risk based on one or more of whether the range of motion of the surgical instrument has exceeded a preset threshold value and whether the surgical instrument corresponds to a preset singularity region. Here, the singularity may include a kinematic singularity, and the range of motion may include a movable angle of the surgical instrument.

Meanwhile, when the range of motion is considered, the processor 2011 may determine the presence of a risk based on whether the range of motion of the surgical instrument exceeds the preset threshold value. Specifically, when the presence of a risk is determined based on the range of motion, the processor 2011 may calculate expected driving information based on initial driving information of the surgical instrument and motion range information of the surgical instrument, calculate reference driving information based on the motion range information, and determine the presence of a risk associated with the range of motion of the surgical instrument based on the expected driving information, the motion range information, and the first driving information.

The preset threshold value refers to a value related to a movable angle set according to the surgical instrument. For example, the set movable angle of the end tool may be 90°. When the range of motion of the surgical instrument exceeds the preset threshold value, the surgical instrument may be damaged due to mechanical constraints of the surgical instrument or the patient's body may be damaged due to excessive motion.

For example, the processor 2011 may determine the presence of a risk according to Equation 5 below based on whether the range of motion of the surgical instrument has exceeded the preset threshold value.

$$( True , false ) = {f}_{3} ( {X}_{robot\_delta} , J ( {q}_{curr} ) , {q}_{curr} , {q}_{upper\_limit} , {q}_{lower\_limit} ) \qquad [ Equation 5 ]$$

According to Equation 5, the processor 2011 may determine the presence of a risk “True, false” based on a function ƒ₃, difference information X_{robot_delta}, initial driving information q_{curr} of the surgical instrument, upper limit information q_{upper_limit} of the range of motion of the surgical instrument, lower limit information q_{lower_limit} of the range of motion of the surgical instrument, a Jacobian matrix J(q_{curr}) for the initial driving information q_{curr}.

The difference information X_{robot_delta} includes a difference between position or orientation information of the surgical instrument before driving the surgical instrument based on the first driving information and position or orientation information of the surgical instrument after driving the surgical instrument based on the first driving information. In an example, the difference information X_{robot_delta} may be calculated based on the first driving information. In another example, the difference information X_{robot_delta} may be calculated based on the first driving information and the initial driving information. The difference information X_{robot_delta} may be in the form of a homogeneous transform matrix or a screw, but the present disclosure is not limited thereto.

The initial driving information q_{curr} includes one or more of initial position information or initial orientation information of the surgical instrument before driving the surgical instrument based on the first driving information. Meanwhile, the initial driving information q_{curr} may be a vector of n dimensions (where n=mechanical degrees of freedom of the surgical instrument), but the present disclosure is not limited thereto.

The motion range information of the surgical instrument includes the upper limit information q_{upper_limit} and the lower limit information q_{lower_limit} of the range of motion of the surgical instrument. The upper limit information q_{upper_limit} of the range of motion of the surgical instrument includes upper limit information of a range of motion of the joints included in the surgical instrument. The lower limit information q_{lower_limit} of the range of motion of the surgical instrument includes lower limit information of the range of motion of the joints included in the surgical instrument.

The Jacobian matrix J(q_{curr}) may include a Jacobian matrix calculated based on the initial driving information q_{curr}.

Meanwhile, the function ƒ₃ may be implemented according to Equations 6 to 8 as follows.

Specifically, the processor 2011 may calculate expected driving information z according to Equation 6 below.

$$z = \frac{π ( 2 {q}_{c u r r} - {q}_{upper\_limit} - {q}_{lower\_limit} )}{2 ( {q}_{upper\_limit} - {q}_{lower\_limit} )} \qquad [ Equation 6 ]$$

According to Equation 6, the processor 2011 may calculate the expected driving information z based on the initial driving information q_{curr}, the upper limit information q_{upper_limit} of the range of motion of the surgical instrument, and the lower limit information q_{lower_limit} of the range of motion of the surgical instrument.

In addition, the processor 2011 may obtain reference driving information γ of n dimensions (where n=mechanical degrees of freedom of the surgical instrument) for determining whether the range of motion of the surgical instrument exceeds the preset threshold value, according to Equation 7 below.

$$γ = \sqrt{\frac{{q}_{upper \_ limit} - {q}_{lower \_ limit}}{πε} - 1} \qquad [ Equation 7 ]$$

According to Equation 7, the processor 2011 may obtain the reference driving information γ of n dimensions (where n=mechanical degrees of freedom of the surgical instrument) based on the upper limit information q_{upper_limit} of the range of motion of the surgical instrument and the lower limit information glower limit of the range of motion of the surgical instrument.

In addition, the processor 2011 may determine the presence of a risk according to Equation 8 below based on whether the range of motion of the surgical instrument has exceeded the preset threshold value. For example, through Equation 8 below, the processor 2011 may determine that the range of motion of the surgical instrument has exceeded a maximum movable angle or 90° when a true value is obtained, and may determine that the range of motion of the surgical instrument has not exceeded the maximum movable angle or 90° when a false value is obtained.

$$\vert {z}_{i} \vert > {γ}_{i} and {{z}_{i} ( {J}^{+} ( {q}_{curr} ) {X}_{robot \_ delta} )}_{i} > 0 ( 0 < i \leq n ) \qquad [ Equation 8 ]$$

According to Equation 8, the processor 2011 may determine the presence of a risk based on whether the range of motion of the surgical instrument has exceeded the preset threshold value, which is obtained based on the difference information X_{robot_delta}, the Jacobian matrix J⁺(q_{curr}), the expected driving information z, and the reference driving information γ. Here, an addition (+) operation refers to a pseudo-inverse matrix. In addition, n denotes the number of elements included in the vector. The processor 2011 may obtain a true value when one of the n elements included in the vector satisfies Equation 8. For example, when the range of motion of the surgical instrument exceeds the preset threshold value, which satisfies Equation 8 and thus the processor 2011 may obtain a true value, and when the range of motion of the surgical instrument does not exceed the preset threshold value, the processor 2011 may obtain a false value.

Meanwhile, when the kinematic singularity is considered, the processor 2011 may determine the presence of a risk based on whether the surgical instrument corresponds to the preset singularity region. Specifically, when the processor 2011 determines the presence of a risk based on the singularity region, the processor 2011 may calculate manipulability information related to the first driving information and manipulability information related to the singularity region based on the first driving information, determine the presence of a risk associated with the singularity region of the surgical instrument based on the manipulability information related to the first driving information and the manipulability information related to the singularity region.

For example, the processor 2011 may determine the presence of a risk according to Equations 9 and 10 below based on whether the surgical instrument corresponds to the preset singularity region.

First, the processor 2011 may calculate manipulability information related to the first driving information and manipulability information related to the singularity region according to Equation 9 below based on the first driving information.

$$MI = \sqrt{det ( J ( {q}_{curr} ) {J}^{T} ( {q}_{curr} ) )} \qquad [ Equation 9 ]$$

According to Equation 9, the processor 2011 may obtain manipulability information MI related to the first driving information based on the Jacobian matrix J(q_{curr}). The manipulability information MI may include a manipulability factor value for determining whether the surgical instrument corresponds to the preset singularity region. In an example, when the manipulability factor value included in the manipulability information MI is less than or equal to the preset threshold value, the processor 2011 may determine that the surgical instrument corresponds to the preset singularity region and thus it is dangerous. In another example, when the manipulability factor value included in the manipulability information MI becomes “0,” the processor 2011 may determine that the surgical instrument corresponds to the preset singularity region and thus it is dangerous.

In addition, the processor 2011 may determine the presence of a risk associated with the singularity region of the surgical instrument based on the manipulability information related to the first driving information and the manipulability information related to the singularity region.

$$k ( MI , \overline{MI} ) = \{ \begin{matrix}0 , & \overline{MI} \leq MI \\ 4 ( \frac{4 {MI}^{3} - 9 {MI}^{2} \overline{MI} + 6 {\overline{MI}}^{2} MI - {\overline{MI}}^{3}}{{\overline{MI}}^{3}} ) , & \overline{MI} / 2 < MI < \overline{MI} \\ 1 , & MI \leq \overline{MI} / 2\end{matrix} \qquad [ Equation 10 ]$$

According to Equation 10, the processor 2011 may determine whether the range of motion of the surgical instrument includes the preset singularity region, based on a shape function k, manipulability information M̅I̅ related to the singularity region, and the manipulability information MI related to the first driving information.

As the manipulability information M̅I̅ related to the singularity region approaches “0,” the range of motion of the surgical instrument according to the driving information may be changed at a position close to the singularity, allowing the user to manipulate the surgical instrument more intuitively, but there is a high possibility that the range of motion may easily fall into the singularity due to numerical errors or the like. As the manipulability information related to the singularity region is far from “0,” there is a small possibility that the range of motion may fall into the singularity due to numerical errors or the like, but, since the position is not close to the singularity, it may be difficult to make a situation allowing the user to manipulate the surgical instrument more intuitively by changing the motion range information of the surgical instrument according to the driving information.

Meanwhile, the processor 2011 may update the first driving information based on the result of determining the presence of the risk. In an example, when it is determined that the range of motion of the surgical instrument exceeds the preset threshold value and thus it is dangerous, the processor 2011 may update the first driving information such that the range of motion of the surgical instrument is included within the preset threshold value. In another example, when it is determined that the range of motion of the surgical instrument corresponds to the preset singularity region and thus it is dangerous, the processor 2011 may update the first driving information such that the range of motion of the surgical instrument does not correspond to the preset singularity region.

Meanwhile, the processor 2011 may update the first driving information based on the motion range information of the surgical instrument. For example, the processor 2011 may update the first driving information so that the range of motion of the surgical instrument corresponds to an upper or lower limit of the range of motion of the joints included in the surgical instrument. As used herein, the joints refer to any joints included in the surgical instrument, and may include joints that perform a pitch motion, a yaw motion, a roll motion, and the like of the end tool, as well as joints that determine the position of the RCM of the surgical instrument. However, examples of the joints are not limited to the above description.

Meanwhile, the processor 2011 may update the driving information such that the range of motion of the surgical instrument according to the driving information does not include the preset singularity region. For example, the processor 2011 may update the driving information by removing information, which causes the motion of the surgical instrument to enter the singularity region, from the driving information.

Meanwhile, the processor 2011 may generate compensation driving information based on the initial driving information of the surgical instrument, the first driving information, and vector information related to the singularity region, and update the first driving information based on the initial driving information of the surgical instrument, the first driving information, the manipulability information related to the first driving information, the manipulability information related to the singularity region, and the compensation driving information. For example, when the processor 2011 obtains a non-zero value according to Equation 10 described above, the processor 2011 may generate compensation driving information X_{correction} based on the initial driving information of the surgical instrument, the first driving information, and the vector information related to the singularity region.

Meanwhile, in generating the compensation driving information X_{correction}, the processor 2011 may update the first driving information by changing one or more of the position information and the orientation information of the surgical instrument included in the first driving information.

In an example, the processor 2011 may update the first driving information by changing both the position information and the orientation information of the surgical instrument included in the first driving information.

In this case, the processor 2011 may obtain updated difference information X_{safe_robot_delta} by further performing a coefficient-related operation according to Equation 11 below in order to prevent a case, in which the range of motion of the surgical instrument enters the singularity region due to an error in a numerical analysis method, through Equation 11 below.

$${X}_{safe \_ robot \_ delta} = \frac{1 - sign ( {X}_{robot \_ delta} \cdot {n}_{m} )}{2} k ( MI , \overline{MI} ) {X}_{correction} + k ( MI , \overline{MI} / 2 ) {n}_{m} \qquad [ Equation 11 ]$$

$$where {n}_{m} = \frac{{( \frac{\partial MI}{\partial q} {J}^{+} ( {q}_{curr} ) )}^{T}}{‖ \frac{\partial MI}{\partial q} {J}^{+} ( {q}_{curr} ) ‖} , {X}_{correction} = {X}_{robot \_ delta} \cdot {n}_{m}$$

According to Equation 11, the processor 2011 may obtain the updated difference information X_{safe_robot_delta} on the basis of the difference information X_{robot_delta} based on the first driving information, the initial driving information q_{curr} of the surgical instrument, the manipulability information MI related to the first driving information, the manipulability information M̅I̅ related to the singularity region, and the compensation driving information X_{correction}.

The updated difference information X_{safe_robot_delta} includes a difference between position or orientation information of the surgical instrument before driving the surgical instrument on the basis of the updated first driving information, and position or orientation information of the surgical instrument after driving the surgical instrument on the basis of the orientation information and the updated first driving information. The updated difference information X_{safe_robot_delta} may be in the form of a homogeneous transform matrix or a screw, but the present disclosure is not limited thereto.

In another example, the processor 2011 may update the first driving information by changing only the position information of the surgical instrument included in the first driving information. Specifically, the processor 2011 may generate the compensation driving information based on the initial driving information of the surgical instrument, the first driving information, and the vector information related to the singularity region.

First, the processor 2011 may generate the compensation driving information X_{correction} according to Equation 12 below in order to change only the position information of the surgical instrument among the information included in the driving information.

$${X}_{correction} = {[ \begin{matrix}{x}^{*} & {y}^{*} & {z}^{*} & 0 & 0 & 0\end{matrix} ]}^{T} \qquad [ Equation 12 ]$$

According to Equation 12, the processor 2011 may obtain the compensation driving information X_{correction} based on a position value x* of the surgical instrument related to the x-axis, a position value y* of the surgical instrument related to the y-axis, and a position value z* of the surgical instrument related to the z-axis.

The compensation driving information X_{correction} is a vector in the form of a screw, and may include six elements. For example, a first element of the compensation driving information X_{correction} may be the position value x* of the surgical instrument related to the x-axis, a second element thereof may be the position value y* of the surgical instrument related to the y-axis, a third element thereof may be the position value z* of the surgical instrument related to the z-axis, a fourth element thereof may be a directional rotation angle ϕᵣ* related to the roll motion of the surgical instrument, a fifth element thereof may be a directional rotation angle ϕₚ* related to the pitch motion of the surgical instrument, a sixth element thereof may be a directional rotation angle ϕ_{y}* related to the yaw motion of the surgical instrument. However, examples of the compensation driving information are not limited to the above description.

Here, the position value x* of the surgical instrument related to the x-axis, the position value y* of the surgical instrument related to the y-axis, the position value z* of the surgical instrument related to the z-axis may be obtained using a solution of an optimization problem to be described in detail below.

The optimization problem is based on the difference information X_{robot_delta} and elements (a, b, c, and d extracted from a surface vector representing boundaries of the singularity region according to Equation 13 below.

$$Let {( \frac{\partial MI}{\partial q} {J}^{+} ( {q}_{curr} ) )}^{T} = S ( q ) , \qquad [ Equation 13 ]$$

then a=S(q)₁, b=S(q)₂, c=S(q)₃, d=S(q)·X_{robot_delta}

In addition, the processor 2011 may obtain updated difference information X_{safe_robot_delta} by subtracting the difference information X_{robot_delta} from the compensation driving information X_{correction}. This may be understood as a process of removing orientation-related vector components, which cause the motion of the surgical instrument to enter the singularity region, from the existing first driving information or difference information.

Meanwhile, in order to prevent the range of motion of the surgical instrument from falling into the singularity region due to an error of the numerical analysis method, the processor 2011 may obtain the updated difference information X_{safe_robot_delta} by further performing a coefficient-related operation according to Equation 14 below.

$${X}_{safe \_ robot \_ delta} = \frac{1 - sign ( {X}_{robot \_ delta} \cdot {n}_{m} )}{2} k ( MI , \overline{MI} ) {X}_{correction} + k ( MI , \overline{MI} / 2 ) {{n}_{m}}^{p} \qquad [ Equation 14 ]$$

$$where {n}_{m} = \frac{{( \frac{\partial MI}{\partial q} {J}^{+} ( {q}_{curr} ) )}^{T}}{‖ \frac{\partial MI}{\partial q} {J}^{+} ( {q}_{curr} ) ‖} , {{n}_{m}}^{p} = \frac{{X}_{correction}}{‖ {X}_{correction} ‖}$$

According to Equation 14, the processor 2011 may obtain the updated difference information X_{safe_robot_delta} on the basis of the difference information X_{robot_delta} based on the first driving information, the initial driving information q_{curr} of the surgical instrument, the manipulability information MI related to the first driving information, the manipulability information M̅I̅ related to the singularity region, and the compensation driving information X_{correction}.

Meanwhile, the processor 2011 may update the first driving information by changing only the orientation information of the surgical instrument included in the first driving information. Specifically, the processor 2011 may update the first driving information by fixing the position information of the surgical instrument included in the first driving information and changing only the orientation information.

First, the processor 2011 may generate compensation driving information related to the orientation information of the surgical instrument based on the initial driving information of the surgical instrument, the first driving information, and the vector information related to the singularity region.

In addition, the processor 2011 may update the first driving information based on the initial driving information of the surgical instrument, the first driving information, the manipulability information related to the first driving information, the manipulability information related to the singularity region, and the compensation driving information related to the orientation information of the surgical instrument.

The compensation driving information may be information including a compensation value that serves as a reference for updating the first driving information to escape from the risk by changing only motion orientation of the motion of the surgical instrument. For example, the compensation driving information may be for changing only the orientation information of the surgical instrument.

When the surgical instrument is driven with the updated first driving information, only the motion orientation of the surgical instrument may be changed compared to when the surgical instrument is driven with the first driving information that is not updated. In this case, by changing only the motion orientation of the surgical instrument, a situation causing a mechanical risk to the surgical robot and a situation causing damage to the human body may be prevented in advance.

For example, the processor 2011 may generate the compensation driving information X_{correction} according to Equation 15 below, such that the range of motion of the surgical instrument does not include the singularity region.

$${X}_{correction} = {[ \begin{matrix}0 & 0 & 0 & {{ϕ}_{r}}^{*} & {{ϕ}_{p}}^{*} & {{ϕ}_{y}}^{*}\end{matrix} ]}^{T} \qquad [ Equation 15 ]$$

According to Equation 15, the processor 2011 may generate the compensation driving information X_{correction} in the form of a screw. Here, a value of each of the directional rotation angle ϕᵣ* related to the roll motion of the surgical instrument, the directional rotation angle ϕₚ* related to the pitch motion of the surgical instrument, and the directional rotation angle ϕ_{y}* related to the yaw motion of the surgical instrument may be obtained by using one solution of the optimization problem, which will be described in detail below.

The optimization problem is based on θ, which is the directional rotation angle ϕᵣ* related to the roll motion of the surgical instrument, the initial driving information q_{curr} of the surgical instrument, and the difference information X_{robot_delta}, and the elements a, b, c, and d, which are extracted from the surface vector representing the boundaries of the singularity region, according to Equation 16 below.

$$Let {( \frac{\partial MI}{\partial q} {J}^{+} ( {q}_{curr} ) )}^{T} = S ( q ) , \qquad [ Equation 16 ]$$

then a=S(q)₄, b=S(q)₅, c=S(q)₆, d=S(q)·X_{robot_delta}

Meanwhile, when the processor 2011 modifies the directional rotation angle ϕ_{y}* related to the yaw motion of the surgical instrument, the optimization problem may be solved according to Equation 17 below.

$$minimize {{ϕ}_{r}}^{2} + {{ϕ}_{p}}^{2} + {{ϕ}_{y}}^{2} \qquad [ Equation 17 ]$$

$$subject to a {ϕ}_{r} + b {ϕ}_{p} + c {ϕ}_{y} + d = 0$$

$${ϕ}_{p} : {ϕ}_{y} = - sin θ : cos θ$$

Meanwhile, when the processor 2011 modifies the directional rotation angle ϕₚ* related to the pitch motion of the surgical instrument, the optimization problem may be solved according to Equation 18 below.

$$minimize {{ϕ}_{r}}^{2} + {{ϕ}_{p}}^{2} + {{ϕ}_{y}}^{2} \qquad [ Equation 18 ]$$

$$subject to a {ϕ}_{r} + b {ϕ}_{p} + c {ϕ}_{y} + d = 0$$

$${ϕ}_{p} : {ϕ}_{y} = cos θ : sin θ$$

Meanwhile, the processor 2011 may obtain the updated difference information X_{safe_robot_delta} by subtracting the difference information X_{robot_delta} from the compensation driving information X_{correction}.

Meanwhile, in order to prevent the range of motion of the surgical instrument from falling into the singularity region due to an error of the numerical analysis method, the processor 2011 may obtain the updated difference information X_{safe_robot_delta} by further performing a coefficient-related operation according to Equation 19 below.

$${X}_{safe \_ robot \_ delta} = \frac{1 - sign ( {X}_{robot \_ delta} \cdot {n}_{m} )}{2} k ( MI , \overline{MI} ) {X}_{correction} + k ( MI , \overline{MI} / 2 ) {{n}_{m}}^{o} \qquad [ Equation 19 ]$$

$$where {n}_{m} = \frac{{( \frac{\partial MI}{\partial q} {J}^{+} ( {q}_{curr} ) )}^{T}}{‖ \frac{\partial MI}{\partial q} {J}^{+} ( {q}_{curr} ) ‖} , {{n}_{m}}^{o} = \frac{{X}_{correction}}{‖ {X}_{correction} ‖}$$

According to Equation 19, the processor 2011 may obtain the updated difference information X_{safe_robot_delta} on the basis of the difference information X_{robot_delta} based on the first driving information, the initial driving information q_{curr} of the surgical instrument, the manipulability information MI related to the first driving information, the manipulability information M̅I̅ related to the singularity region, and the compensation driving information X_{correction}.

According to an embodiment of the present disclosure, the first driving information may be updated by changing only the orientation information of the surgical instrument included in the first driving information, so that the motion of the surgical instrument can be changed within a range that does not deviate significantly from the existing expected motion, thereby facilitating the prediction of the motion and helping to perform safe surgery.

Meanwhile, the processor 2011 may update the driving information so that the range of motion of the surgical instrument is included within the preset threshold value and the range of motion of the surgical instrument according to the driving information does not include the preset singularity region.

Referring to FIG. 21 again, in operation 2140, the processor 2011 drives the surgical instrument based on the result of determining the presence of the risk. In an example, when driving the surgical instrument based on the calculated first driving information is determined to be dangerous, the processor 2011 may not drive the surgical instrument. In this case, the processor 2011 may update the first driving information based on the result of determining the presence of the risk, and then drive the surgical instrument based on the updated first driving information. In another example, when driving the surgical instrument based on the calculated first driving information is determined not to be dangerous, the processor 2011 may drive the surgical instrument based on the first driving information.

Meanwhile, the processor 2011 may calculate second driving information based on the first driving information, and may drive the surgical instrument based on the second driving information.

In an example, the processor 2011 may calculate the second driving information by performing an inverse kinematic transformation according to Equation 20 below.

$${q}_{robot \_ target} = IK ( {T}_{robot \_ target} ) \qquad [ Equation 20 ]$$

According to Equation 20, the processor 2011 obtains second driving information q_{robot_target} based on an inverse kinematic transformation function IK and the first driving information T_{robot_target}. Similarly, the processor 2011 may obtain the second driving information q_{robot_target} based on the inverse kinematic transformation function IK and the updated first driving information.

The inverse kinematic transformation function IK includes a function that transforms a vector defined with respect to a motion of the member, which allows a user to manipulate the position and function of the surgical instrument, into a vector defined with respect to a motion of the surgical instrument. For example, the inverse kinematic transformation function IK may include a function that transforms a vector defined in a task space of the multi-joint type surgical instrument into a vector defined in a joint space of the surgical instrument. Here, the vector defined in the task space may include a 16-dimensional homogeneous transform matrix, and the vector defined in the joint space may include n-dimensional mechanical degrees of freedom of the multi-joint type surgical instrument.

Specifically, in order to improve computational speed, the processor 2011 may transform the homogeneous transform matrix into a six-dimensional value in the form of a screw including three-dimensional position and orientation information. In this case, in order to perform the inverse kinematic transformation, the processor 2011 may use a numerical analysis method, such as the Newton-Raphson numerical inverse kinematics method, but the present disclosure is not limited thereto.

In another example, the processor 2011 may calculate the second driving information by performing an inverse kinematic transformation according to Equation 21 below.

$${q}_{robot \_ target} = IK ( {X}_{robot \_ delta} ) \qquad [ Equation 21 ]$$

According to Equation 21, the processor 2011 obtains the second driving information q_{robot_target} based on the inverse kinematic transformation function IK and the difference information X_{robot_delta}. Similarly, the processor 2011 may obtain the second driving information q_{robot_target} based on the inverse kinematic transformation function IK and the updated difference information.

Meanwhile, the first driving information and the difference information are distinguished from each other and described for convenience of description, but the difference information may be information included in the first driving information. According to an embodiment of the present disclosure, the processor 2011 may generate manipulation information related to a user's motion for driving the surgical instrument, and calculate first driving information based on the manipulation information. In this case, the first driving information may include position and orientation information of the surgical instrument. In addition, the first driving information may include difference information indicating a difference between position or orientation information of the surgical instrument before driving the surgical instrument based on the first driving information and position or orientation information of the surgical instrument after driving the surgical instrument based on the orientation information and the first driving information. In addition, the processor 2011 may determine the presence of a risk associated with a motion of the surgical instrument based on the difference information included in the first driving information. In addition, the processor 2011 may update the difference information included in the first driving information based on the result of determining the presence of the risk. In addition, the processor 2011 may drive the surgical instrument based on the result of determining the presence of the risk.

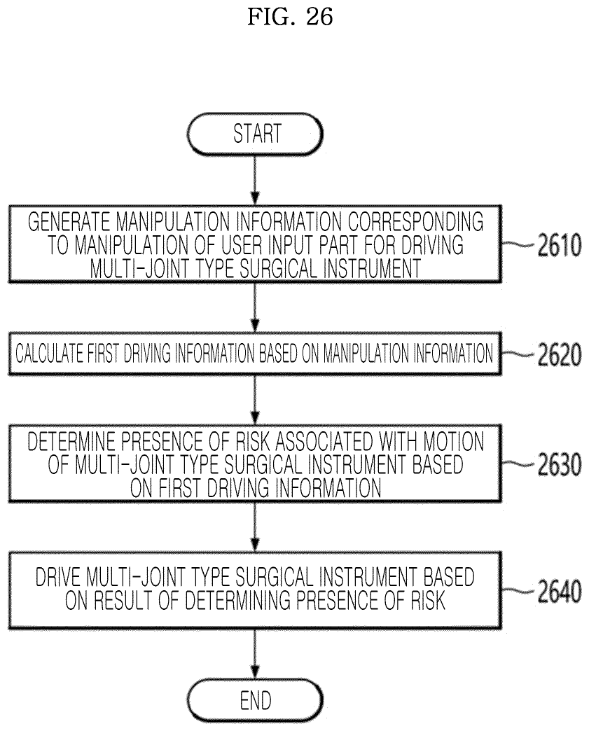

Hereinafter, another example of the method of driving a surgical instrument is described with reference to FIG. 26.

FIG. 26 is a flowchart for describing another example of the method of driving the surgical instrument according to an embodiment.

Operations 2610 to 2640 of FIG. 26 corresponds to operations 2110 to 2140 of FIG. 21, respectively. Thus, a description of redundant content of operations 2610 to 2640 will be omitted.

In operation 2610, the processor 2011 generates manipulation information corresponding to manipulation of the user input part for driving the multi-joint type surgical instrument. For example, when a user manipulates the user input part to positionally move the robot arm unit or the end tool of the slave robot, or to manipulate a surgical motion, the processor 2011 may generate manipulation information corresponding to the user's manipulation for the user input part.

Meanwhile, the processor 2011 may generate the manipulation information based on position and orientation information of the user input part of the multi-joint type surgical instrument.

In operation 2620, the processor 2011 calculates first driving information based on the manipulation information. Specifically, the processor 2011 generates first driving information related to a motion of the multi-joint type surgical instrument based on the manipulation information.

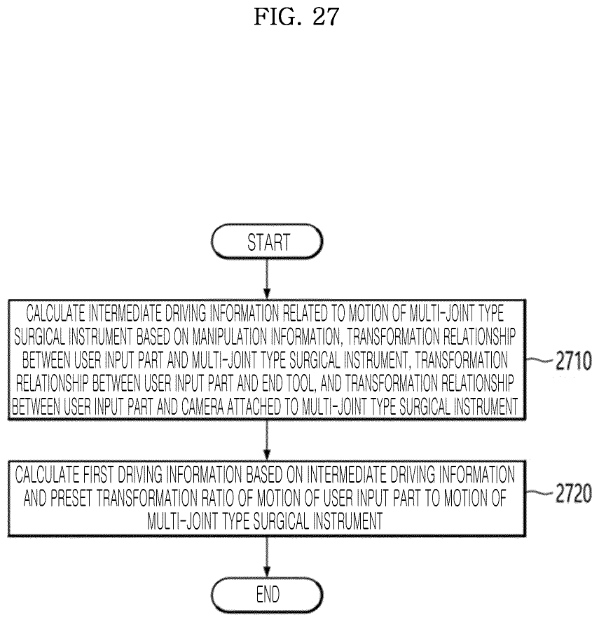

Hereinafter, another example of a method by which the processor calculates the first driving information according to an embodiment will be described with reference to FIG. 27.

FIG. 27 is a flowchart for describing another example of the method by which the processor according to an embodiment calculates the first driving information.

Operations 2710 and 2720 of FIG. 27 correspond to operations 2210 and 2220 of FIG. 22, respectively. Thus, a description of redundant content of operations 2710 and 2720 will be omitted.

In operation 2710, the processor 2011 calculates intermediate driving information related to a motion of the multi-joint type surgical instrument based on manipulation information, a transformation relationship between the user input part and the multi-joint type surgical instrument, a transformation relationship between the user input part and the end tool, and a transformation relationship between the user input part and the camera attached to the multi-joint type surgical instrument.

Specifically, the processor 2011 calculates intermediate position information of the multi-joint type surgical instrument based on a transformation relationship between the user input part and the camera attached to the multi-joint type surgical instrument, calculates intermediate orientation information of the surgical instrument based on a transformation relationship between the user input part and the multi-joint type surgical instrument, and generates intermediate driving information by using the intermediate position information of the multi-joint type surgical instrument and the intermediate orientation information of the multi-joint type surgical instrument.

For example, according to Equation 2 described above, the processor 2011 may generate intermediate driving information T_{robot} related to a motion of the multi-joint type surgical instrument based on a function ƒ₁, manipulation information T_{device}, a rotation matrix R_{device→robot_global} that transforms from a coordinate system of the user input part into a coordinate system of the multi-joint type surgical instrument, a rotation matrix R_{device→robot_tool} that transforms from a coordinate system of the user input part into a coordinate system of the end tool, and a rotation matrix R_{robot_global→camera_view} that transforms from a coordinate system of the multi-joint type surgical instrument into a coordinate system of the multi-joint type surgical instrument according to the camera attached to the multi-joint type surgical instrument.

In operation 2720, the processor 2011 calculates first driving information based on the intermediate driving information and a preset transformation ratio of a motion of the user input part to a motion of the multi-joint type surgical instrument.

Specifically, the processor 2011 calculates position information of the multi-joint type surgical instrument based on the intermediate position information of the multi-joint type surgical instrument included in the intermediate driving information and a preset position transformation ratio, calculates orientation information of the multi-joint type surgical instrument based on the intermediate orientation information of the multi-joint type surgical instrument included in the intermediate driving information and a preset orientation transformation ratio, and generates the first driving information by using the position information of the multi-joint type surgical instrument and the orientation information of the multi-joint type surgical instrument.

Here, the first driving information may be calculated based on the preset transformation ratio of the motion of the user input part to the motion of the multi-joint type surgical instrument, which is related to one or more of a pitch motion, a yaw motion, a roll motion, and an actuation motion of the end tool.

Referring to FIG. 26 again, in operation 2630, the processor 2011 determines the presence of a risk associated with a motion of the multi-joint type surgical instrument based on the first driving information. In an example, the processor 2011 may determine the presence of a risk associated with at least one of the pitch motion, the yaw motion, the actuation operation, and the roll motion of the end tool included in the multi-joint type surgical instrument, based on the first driving information. In another example, the processor 2011 may determine the presence of a risk associated with a motion of the joint involved in the RCM included in the multi-joint type surgical instrument, based on the first driving information.

Meanwhile, the processor 2011 may determine the presence of a risk based on one or more of whether a range of motion formed by one or more joints of the multi-joint type surgical instrument has exceeded a preset threshold value and whether the multi-joint type surgical instrument corresponds to a preset singularity region.

Meanwhile, when the presence of a risk is determined based on the range of motion, the processor 2011 may calculate expected driving information based on initial driving information of the multi-joint type surgical instrument and motion range information of the multi-joint type surgical instrument, calculate reference driving information based on the motion range information, and determine the presence of a risk associated with the range of motion formed by one or more joints of the multi-joint type surgical instrument based on the expected driving information, the motion range information, and the first driving information.

Meanwhile, when the presence of a risk is determined based on the singularity region, the processor 2011 may calculate manipulability information related to the first driving information and manipulability information related to the singularity region based on the first driving information, determine the presence of a risk associated with the singularity region of the multi-joint type surgical instrument based on the manipulability information related to the first driving information and the manipulability information related to the singularity region.

Meanwhile, the processor 2011 may update the first driving information based on the result of determining the presence of the risk.

Meanwhile, the processor 2011 may update the first driving information based on the motion range information of the multi-joint type surgical instrument.

Meanwhile, the processor 2011 may generate compensation driving information based on the initial driving information of the multi-joint type surgical instrument, the first driving information, and vector information related to the singularity region, and update the first driving information based on the initial driving information of the multi-joint type surgical instrument, the first driving information, the manipulability information related to the first driving information, the manipulability information related to the singularity region, and the compensation driving information.

Meanwhile, the processor 2011 may update the first driving information by changing only the orientation information of the multi-joint type surgical instrument included in the first driving information.

In operation 2640, the processor 2011 drives the multi-joint type surgical instrument based on the result of determining the presence of the risk. For example, the processor 2011 may implement a pitch motion, a yaw motion, and an actuation motion of the end tool of the multi-joint type surgical instrument within a safe motion range based on the updated driving information,

Meanwhile, the processor 2011 may calculate second driving information based on the first driving information, and may drive the multi-joint type surgical instrument based on the second driving information. For example, the processor 2011 may calculate the second driving information by performing an inverse kinematic transformation based on the first driving information, and may drive the multi-joint type surgical instrument based on the calculated second driving information.

According to the above description, the processor 2011 may generate manipulation information related to a user's motion for driving the surgical instrument, calculate first driving information based on the manipulation information, determine the presence of a risk associated with a motion of the surgical instrument based on the first driving information, and drive the surgical instrument based on the result of determining the presence of the risk, so that the processor 2011 may more accurately reflect an intuitive motion of the user and perform surgery through the surgical robot. In addition, the processor 2011 may prevent in advance a situation that causes a mechanical risk to the surgical robot and a situation that causes damage to the human body.

The above-described method may be recorded as a program that may be executed on a computer, and may be implemented in a general-purpose digital computer operating the program using a computer-readable recording medium. In addition, the structure of the data used in the method described above may be recorded on a computer-readable recording medium through various means. Examples of the computer-readable recording medium include storage media such as magnetic storage media (e.g., ROM, floppy disks, hard disks, and the like), and optical read media (e.g., CD-ROMs, DVDs, and the like).

Meanwhile, the above-described method may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smart phones) directly. When distributed online, at least a part of the computer program product may be temporarily generated or at least temporarily stored in the machine-readable storage medium, such as memory of the manufacturer's server, a server of the application store, or a relay server.

It will be understood by those skilled in the art to which the present embodiment pertains that the present disclosure may be implemented in modified forms without departing from the spirit and scope of the present disclosure. Therefore, the disclosed methods should be considered in an illustrative aspect rather than a restrictive aspect. The scope of the present disclosure should be defined by the claims rather than the above-mentioned description, and equivalents to the claims should be interpreted to fall within the present disclosure.

According to the above-described technical solutions of the present disclosure, a surgical robot according to the present disclosure can more accurately reflect a user's intuitive manipulation and perform surgery, by generating manipulation information related to a user's motion for driving a surgical instrument, generating driving information related to a motion of the surgical instrument and inverse kinematic transformation information based on the manipulation information, determining the presence of a risk associated with the motion of the surgical instrument by using the driving information and the inverse kinematic transformation information, and updating the driving information based on the presence of the risk.

Further, in the present disclosure, a situation causing a mechanical risk to a surgical robot and a situation causing damage to a human body can be prevented in advance.

The effects of the present disclosure are not limited to those mentioned above, and other effects not mentioned may be clearly understood by those of ordinary skill in the art from the above description.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of driving a surgical instrument, the method comprising:
generating manipulation information related to a motion of a user for driving the surgical instrument;
calculating first driving information based on the manipulation information;
determining a presence of a risk associated with a motion of the surgical instrument based on the first driving information; and
driving the surgical instrument based on a result of the determining the presence of the risk.

2. The method of claim 1, wherein the generating of the manipulation information includes generating the manipulation information based on position and orientation information of a member that allows the user to manipulate a position and a function of the surgical instrument.

3. The method of claim 1, wherein
the calculating the first driving information comprises:
calculating intermediate driving information based on the manipulation information and a transformation relationship between the motion of the user and the motion of the surgical instrument; and
calculating the first driving information based on the intermediate driving information and a preset transformation ratio of the motion of the user to the motion of the surgical instrument.

4. The method of claim 3, wherein
the calculating the intermediate driving information comprises:
calculating intermediate position information of the surgical instrument based on a transformation relationship between a member, which allows the user to manipulate a position and a function of the surgical instrument, and a camera attached to the surgical instrument;
calculating intermediate orientation information of the surgical instrument based on a transformation relationship between the member and the surgical instrument; and
generating the intermediate driving information using the intermediate position information of the surgical instrument and the intermediate orientation information of the surgical instrument.

5. The method of claim 3, wherein
the calculating the first driving information comprises:
calculating position information of the surgical instrument based on intermediate position information of the surgical instrument included in the intermediate driving information and a preset position transformation ratio;
calculating orientation information of the surgical instrument based on intermediate orientation information of the surgical instrument included in the intermediate driving information and a preset orientation transformation ratio; and
generating the first driving information using the position information of the surgical instrument and the orientation information of the surgical instrument.

6. The method of claim 1, wherein the determining the presence of the risk comprises determining the presence of the risk based on at least one of whether a range of the motion of the surgical instrument has exceeded a preset threshold value and whether the surgical instrument corresponds to a preset singularity region.

7. The method of claim 6, wherein
when the presence of the risk is determined based on the range of the motion of the surgical instrument, the determining the presence of the risk comprises:
calculating expected driving information based on initial driving information of the surgical instrument and motion range information of the surgical instrument;
calculating reference driving information based on the motion range information; and
determining the presence of the risk associated with the range of motion of the surgical instrument based on the expected driving information, the motion range information, and the first driving information.

8. The method of claim 6, wherein
when the presence of the risk is determined based on the preset singularity region, the determining the presence of the risk comprises:
calculating manipulability information related to the first driving information and manipulability information related to the preset singularity region based on the first driving information; and
determining the presence of the risk associated with the preset singularity region of the surgical instrument based on the manipulability information related to the first driving information and the manipulability information related to the preset singularity region.

9. The method of claim 1, further comprising updating the first driving information based on the result of determining the presence of the risk.

10. The method of claim 9, wherein the updating the first driving information comprises updating the first driving information based on motion range information of the surgical instrument.

11. The method of claim 9, wherein
the updating the first driving information comprises:
generating compensation driving information based on initial driving information of the surgical instrument, the first driving information, and vector information related to a singularity region; and
updating the first driving information based on the initial driving information of the surgical instrument, the first driving information, manipulability information related to the first driving information, manipulability information related to the singularity region, and the compensation driving information.

12. The method of claim 9, wherein the updating the first driving information comprises updating the first driving information by changing only orientation information of the surgical instrument included in the first driving information.

13. The method of claim 1, wherein
the driving the surgical instrument comprises:
calculating second driving information based on the first driving information; and
driving the surgical instrument based on the second driving information.

14. A computing device comprising:
at least one memory; and
at least one processor,
wherein the at least one processor is configured to:
generate manipulation information related to a motion of a user for driving a surgical instrument;
calculate first driving information based on the manipulation information;
determine a presence of a risk associated with a motion of the surgical instrument based on the first driving information; and
drive the surgical instrument based on a result of the determining the presence of the risk.

15. A non-transitory computer-readable recording medium having recorded thereon a program for executing on a computer a method of for driving a surgical instrument, the method comprising:
generating manipulation information related to a motion of a user for driving the surgical instrument;
calculating first driving information based on the manipulation information;
determining a presence of a risk associated with a motion of the surgical instrument based on the first driving information; and
driving the surgical instrument based on a result of the determining the presence of the risk.
